(19) United States
(12) Patent Application Publication
(10) Pub. No.: US 2006/0153865 A1
(43) Pub. Date: Jul. 13, 2006

(54) Antigenic peptides

(76) Inventors: Amir Zakievich Maksyutov, Novosibirskaya obl (RU); Alexander Borisovich Ryzhikov, Novosibirskaya obl (RU); Alexander Alexandrovich Kolobov, Sestroretsk (RU); Zaki Amirovich Maksyutov, Novosibirskaya obl (RU)

Correspondence Address: STEPTOE & JOHNSON LLP, 1330 CONNECTICUT AVENUE, N.W., WASHINGTON, DC, 20036, US

(21) Appl. No.: 10/529,885

(22) PCT Filed: Aug. 19, 2003
(86) PCT No.: PCT/RU03/00421

§ 371 Date: Oct. 5, 2005

(30) Foreign Application Priority Data
Oct. 3, 2002 (RU) 2002-1263396

(51) Int. Cl. A61K 39/21; C12P 21/06; C07H 21/02; C12N 15/867; C07K 14/16; C40B 40/10

(52) U.S. Cl. 424/188.1; 424/189.1; 435/69.1; 435/456; 435/325; 530/350; 536/23.72

(57) ABSTRACT

A method for designing antigenic peptide libraries accounts for naturally occurring and potential variability in a group of protein sequences from a variable pathogen. The peptide libraries can elicit an immune response against a range of pathogen variants.

### FEDERALLY SPONSORED RESEARCH OR DEVELOPMENT

The U.S. Government may have certain rights in this invention pursuant to Department of Health and Human Services Grant Nos. ISTC #2450 and BTEP #34.

### CLAIM OF PRIORITY

This application claims priority to Russian Patent Application Serial No. 2002126396, filed on Sep. 27, 2002, the entire contents of which are hereby incorporated by reference.

### TECHNICAL FIELD

This invention relates to antigenic peptides.

### BACKGROUND

A number of pathogens important to human health are variable pathogens, such as HIV-1, HIV-2, hepatitis B and C, influenza, dengue types 1-4 viruses, malaria, and tuberculosis. These and other variable pathogens present a challenge to vaccine development, due to the extremely high genetic variability of the pathogen. In particular, the number of people infected with HIV throughout the world is growing rapidly, especially in countries with poor health care resources. The development of a highly efficient vaccine could help restrict the propagation of this epidemic. One of the main obstacles to developing such a vaccine is the extremely high genetic diversity of HIV. The genetic diversity is observed in various regions of the world in different subtypes, within a single subtype and even, in some cases, within an individual. This variability results from the high rate of mutations in viral proteins occurring during virus replication. See, for example, Coffin, J. M. Science 1995, 267:483-489; Delwart et al., J. Virol. 1994 68:6672-6683; Markham R. B. et al. Proc. Natl. Acad. Sci. U.S.A. 1998, 95:12568-12573; and McCutchan, F. E. et al. AIDS 1996 10 Suppl 3:S13-S20, each of which is incorporated by reference in its entirety.

It has been shown in animal models that simultaneous exposure to multiple antigenic determinants often induces immune responses to each of them (Thomson, S. A. et al. Proc. Natl. Acad. Sci. U.S.A. 1995, 92:5845-5849; Thomson, S. A. et al. J. Virol. 1998, 72: 2246-2252; and Woodberry, T. et al. J. Virol. 1999, 73:5320-5325, each of which is incorporated by reference in its entirety). This indicates that multiple antigenic determinants are suitable as components of a single vaccine construct.

### SUMMARY

The protective component of an immune response to a variable pathogen is often directed against a variable region of a protein. If, on the other hand, the protective response was directed against a conserved region of a protein, pathogen variability would not present a problem to immunological control of infections and vaccine prophylaxis. However, an immune response against conserved regions of variable pathogens often proves to be nonprotective. A peptide antigen based on a variable region can induce a protective response, but the variability of these regions is so great that it is impractical to cover the variability by addition of individual peptide sequences. A potential vaccine against a variable pathogen should include a variety of antigens, to produce an immune response to the variants of the pathogen to which an individual may become exposed.

Vaccine prophylaxis or therapy against variable infectious agents requires constructing a vaccine capable of inducing wide-range humoral and cellular immune responses to antigen variable regions. A challenge for vaccine prophylaxis and therapy against variable infectious agents is the development of a vaccine capable of inducing an immune response against variable or hypervariable regions of their protein components.

A library of chimeric peptide antigens containing one or more potentially protective epitopes and vaccine constructs containing such antigens can produce a protective immune response. In particular, chimeric peptide libraries are able to induce a protective immune response including both humoral and cellular responses to HIV-1, HIV-2, hepatitis C and B viruses, influenza virus, dengue types 1-4 viruses, malaria, tuberculosis, or other variable pathogens.

Chimeric peptide libraries can be designed to represent naturally occurring or potential variants of protein epitopes. Peptide libraries developed in this manner can produce a broad immune cross-response and can be applied for constructing vaccines, pharmaceuticals, and diagnostic kits. The design methods can be applied to a broad range of antigenic determinants of an infectious agent. Different methods can be applied to one infectious agent or one of its proteins or to a single epitope of the protein.

Immunogenic sets of chimeric peptides, referred to as variable chimeric peptide libraries (VCPLs), represent the naturally occurring and potential variability of antigenically active regions. Variable chimeric peptide libraries are sets of homologous peptides variable at one or more positions. They are designed to mimic the genetic diversity of variable protective determinants of an infectious agent. Such VCPLs can induce production of a wide range of antibodies and cytotoxic T-lymphocytes (CTLs) with a joint specificity that covers the diversity of antigenic variants of the variable infectious agent.

In one aspect, a method of manufacturing a family of antigenic peptides includes locating a plurality of variable positions in a region of a pathogen protein, choosing a peptide sequence of the pathogen protein including the plurality of variable positions, selecting one or more substitute amino acid residues for one of the variable positions based on antigenic similarity to amino acid residues naturally occurring at the variable position of the pathogen protein, and preparing a family of antigenic peptides based on the peptide sequence and including the substitute amino acid residues.

In another aspect, a method of designing a family of peptide sequences includes locating a plurality of variable positions in a region of a pathogen protein, choosing a peptide sequence of the pathogen protein including the plurality of variable positions, and selecting one or more substitute amino acid residues for one of the variable positions of the peptide based on antigenic similarity to amino acid residues naturally occurring at the variable position of the pathogen protein, thereby forming a family of peptide sequences.

Selecting can include determining the antigenic similarity using an antigenic similarity matrix. A frequency can be assigned to each substitute amino acid residue in the family of antigenic peptides. Preparing can include weighting the substitute amino acid residues in the family of antigenic peptides based on the assigned frequency. Assigning can include considering the frequency with which the variations naturally occur. The pathogen protein can include a hypervariable region. The pathogen protein can be associated with a virus. The virus can be HIV, hepatitis B virus or hepatitis C virus, or an influenza. The pathogen protein can be HIV gp120. The region can be the V1, V2, V3, V4, or V5 regions. The pathogen protein can be associated with a malaria pathogen, a dengue pathogen, or a tuberculosis pathogen.

The family of antigenic peptides can include members, such that the members taken together have antigenic similarity to each naturally occurring sequence of the region of the pathogen protein. The family of antigenic peptides can include members, such that the members taken together have antigenic similarity to a non-naturally occurring sequence of the region of the pathogen protein. The method can include identifying peptide sequences of the family, the identified peptide sequences being representative of the sequence diversity of the entire family. Fewer than 500 sequences can be identified as being representative of the sequence diversity of the entire family. Identifying can include calculating a distance between peptide sequences of the family. Calculating a distance can include using an antigenic similarity matrix.

The method can include determining an antigenic similarity between a peptide of the family and a region of a human protein. The method can include removing a peptide from the family of antigenic peptides before preparing the family if the determined antigenic similarity between the peptide of the family and the region of a human protein exceeds a predetermined threshold.

Preparing the family of antigenic peptides can include chemical synthesis of the family of peptides. The chemical synthesis can includes combinatorial synthesis, whereby the peptides are formed as a mixture of different sequences. The separate peptides can be mixed. The chemical synthesis can include parallel synthesis, whereby each peptide is formed separately from other peptides. Preparing the family of antigenic peptides can include expression of the family of peptides by a host organism.

In another aspect, a composition includes a family of antigenic peptides having amino acid sequences having antigenic similarity to amino acid sequences of a variable region of a pathogen protein, wherein each antigenic peptide in the family has at least one amino acid position that varies relative to other antigenic peptides in the family.

One amino acid residue can occur more frequently than another in the position that varies. The family can include greater than 150 or greater than 1,000 mutually unique antigenic peptides. The family can include fewer than 100,000 or fewer than 50,000 mutually unique antigenic peptides. The family can include between 1,000 and 50,000 mutually unique antigenic peptides. The family of antigenic peptides can include sequences having antigenic similarity to sequences from a subtype of HIV. The subtype can be subtype A, subtype B, subtype C, subtype D, subtype F, subtype G, a recombinant subtype, a subtype of HIV group N, a subtype of HIV group O, or combinations thereof. At least two members of the family of antigenic peptides can be mixed together. The family of antigenic peptides can be separated according to sequence. The family can include a multiple antigenic peptide.

In another aspect, a method of eliciting an immune response in a subject includes administering to the subject a composition including a family of antigenic peptides having amino acid sequences having antigenic similarity to amino acid sequences of a variable region of a pathogen protein of a pathogen.

Antigenic similarity can be determined using an antigenic similarity matrix. The composition can be administered to a subject prior to infection by the pathogen. The family of antigenic peptides can have amino acid sequences having antigenic similarity to amino acid sequences of a subtype of the pathogen protein. The family of antigenic peptides can have amino acid sequences having antigenic similarity to amino acid sequences from more than one subtype of the pathogen protein. The composition is administered to a subject infected by the pathogen. The subject can be infected by a subtype of the pathogen. The family of antigenic peptides can have amino acid sequences having antigenic similarity to amino acid sequences from the subtype by which the subject is infected.

In another aspect, a method of diagnosing infection includes contacting a sample with a family of peptides having amino acid sequences having antigenic similarity to amino acid sequences of a variable region of a pathogen protein, wherein each peptide in the family has at least one amino acid position that varies relative to other peptides in the family.

The family of peptides can be antigenically similar to a pathogen subtype, or to more than one pathogen subtype. The family of peptides can be immobilized on a substrate before contacting. The method can include determining if the sample includes antibodies that bind specifically to the family of peptides.

In general, antigenic peptide libraries reflect the existing and potential diversity of a variable region of a pathogen protein. The peptide libraries can be used in prophylactic, therapeutic, and diagnostic applications. The full antigenic diversity of a pathogen epitope can be represented by the library. A library designed to reflect the diversity of all variants of a pathogen can be a broad-spectrum vaccine or diagnostic. A library designed to reflect the diversity one or more subtypes of pathogen can be a subtype-specific vaccine or diagnostic. Peptide sequences that are highly similar to sequences found in human proteins and are thus likely to induce an autoimmune reaction can be removed from the library. A library can be designed for any variable pathogen protein of known sequence. Examples of variable pathogens include HIV-1, HIV-2, hepatitis B and C, influenza, dengue types 1-4 viruses, malaria, and tuberculosis.

The details of one or more embodiments are set forth in the description below. Other features and advantages will be apparent from the description and from the claims.

### DETAILED DESCRIPTION

Much effort has been devoted to development of a vaccine against HIV in particular, but without success to date. To be efficient against the broad range of HIV-1 isolates, potential vaccines should contain immunogens of main isolates representing the entire range of HIV-1 isolates. It is necessary to identify these immunogens and improve their immunogenic properties (McMichael, A. J., and Hanke, T, Nature Medicine 2003, 9:874-880; Baltimore, D. and Heilman, C. Sci. Am. 1998, 279: 98-103; and van der Groen, G. et al. AIDS Res. Hum. Retroviruses 1998, 14 Suppl. 3: S211-S221, each of which is incorporated by reference in its entirety).

Therefore, the resulting vaccine construct should contain key protective B- and T-cell epitopes specific to the broad range of HIV-1 isolates, combined with modern delivery means and adjuvants. Activation of both humoral and cellular branches of the immune system is required (Berzofsky, J. A., and Berkower, I. J., AIDS 1995, 9 Suppl. A: S143-S157, which is incorporated by reference in its entirety). The local immune response on mucous membranes and circulating neutralizing antibodies are considered to play key roles in preventing HIV infection, whereas virus-specific CTLs are likely to eliminate infected cells. See, for example, Lehner, T. et al. Science 1992, 258:1365-1369; and Goulder, P. J. and Walker, B. D. Nature Med 1999, 5:1233-1235, each of which is incorporated by reference in its entirety.

The amino acid sequence of the envelope glycoprotein (also called env or gp120) of HIV is highly variable between independent isolates and between sequential isolates from a single infected individual. The amino acid variability in gp120 is concentrated in specific variable regions, numbered V1-V5. The most variable regions often contain neutralizing epitopes so that the virus partially evades the host's immune response and establishes a persistent infection. The principal neutralizing determinant of gp120 is located in the third hypervariable region (V3).

A number of candidate peptide vaccines against HIV-1 have been developed with the recognition of the V3 region as the principal neutralizing determinant in mind. For example, a method of rational design of a polyvalent subunit vaccine against HIV included determination of neutralizing epitopes of the V2 and V3 domains of HIV gp120 isolates from various areas of the world. See, for example, U.S. Pat. No. 6,042,836, which is incorporated by reference in its entirety. Another approach to synthetic immunogenic HIV-1 peptides included designing a tandem synthetic peptide containing the sequences of T- and B-cell epitopes and the sequence of the V3 loop from various HIV isolates. See, for example, U.S. Pat. No. 5,817,754, which is incorporated by reference in its entirety. However, the high genetic variability of the V3 domain results in a variety of sequences, which cannot be simultaneously introduced into a vaccine construct.

For the same reasons as in the case of vaccines, it is extremely difficult to diagnose antibodies against hypervariable regions of variable infectious agents. These antibodies are the earliest and dominant in the immune response. An example is antibodies against the V3 domain of HIV-1 gp120. See, for example, Baltimore, D. and Heilman, C. Sci. Am. 1998, 279: 98-103, Berzofsky, J. A., and Berkower, I. J., AIDS 1995, 9 Suppl. A: S143-S157, and Carlos, M. P. et al., AIDS Res. Hum. Retroviruses 2000, 16:153-161, each of which is incorporated by reference in its entirety.

The diagnostics of such antibodies against the V3 hypervariable domain of gp120 depends on the genotypes of the antigens involved and the genotype of the infectious agent. It can require using multiple sequences corresponding to a broad range of HIV-1 variants. Otherwise, there is a high risk of missing a rare or modified variant of the virus.

A peptide-based vaccine can include a library of peptides. The peptides can be similar to a variable region of a pathogen protein. In particular, the variable region can include a known antigenic determinant. The peptide sequences in the library are chosen to reflect the naturally occurring and potential variability of the variable region.

Chimeric peptide libraries that mimic the genetic diversity of variable protein regions of a pathogen can induce a protective immune response to a broad spectrum of pathogen variants. To design the chimeric peptide library, a theoretical analysis of protein sequence information is carried out. The theoretical analysis results in a library design that can mimic all known and potential variants of the variable protein region. The library can be a subtype-specific library, mimicking only those variants belonging to a particular subtype of the pathogen, or a broad spectrum library, mimicking variants belonging to more than one subtype. The peptides of the library can bind antibodies that are specific to its various antigenic variants.

In general, the design of an antigenic chimeric peptide library begins by inspecting sequences of a protein from a variable pathogen. A variable region of the protein is identified. The variable region can be a known antigenic determinant. A peptide fragment within the protein sequence including at least one variable position is selected to be the basis of the chimeric peptide library. The fragment includes one or more positions having naturally sequence variations. The peptide fragment can include non-varying positions. The fragment can be 10-50 residues in length, such as 15-45 residues or 20-40 residues in length. For each variable position in the peptide, the residues that occur in that position and the frequency with which each occurs are identified. The choice of residues at each variable position in the library is determined by considerations of antigenic similarity to the residues naturally occurring at that position. The frequency of each residue at a variable position is assigned according to the frequency that it or its corresponding antigenically similar residue occurs naturally. Selection of residues and frequencies is described in more detail below. The residue selection and frequency assignment can be automated, i.e. carried out by a computer program.

The number of sequences in the library (the size of the library) is the product of the number of possible residues at each position in the peptide. The size of the library can include, for example, more than 200,000,000 sequences, fewer than 200,000,000 sequences, fewer than 500,000 sequences, fewer than 200,000 sequences, fewer than I 00,000 sequences, fewer than 50,000 sequences, more than 5,000 sequences, more than 10,000 sequences, or more than 20,000 sequences. The size of the library can be reduced by eliminating the residue that occurs with the lowest frequency until a desired size is reached.

Immunogenic sets of chimeric peptides, (variable chimeric peptide libraries or VCPLs) when taken together, represent the naturally occurring and potential variability of antigenically active regions. Variable chimeric peptide libraries are sets of homologous peptides variable at one or more positions. They are designed to mimic the genetic diversity of main variable protective determinants of an infectious agent. Such VCPLs can induce a wide range of antibodies and cytotoxic T-lymphocytes (CTLs), having a joint specificity that covers the diversity of antigenic variants of the infectious agent. Thus, VCPLs induce humoral and cellular immune responses against the diversity of currently existing variants of an infectious agent and against potential variants which may arise in the future.

Libraries of variable chimeric peptides can be constructed so that the conserved positions of these peptides represent amino acids common for all sequences, and the heterogeneous diversity of the variable positions is mimicked by one or several amino acids. The mimicking amino acids can be chimeric, in other words, they mimic the general heterogeneity of variable positions but need not occur at corresponding positions among the known and potential diversity of natural sequences.

The method of designing VCPLs rests upon comprehensive investigation of the whole body of structural evidence on the organization of immunodominant protein epitopes and on the induction of a protective immune response to the target pathogen. The library design begins with collection and analysis of amino acid sequences from the target pathogen, and estimation of the genetic variability and epidemiological significance of pathogen subtypes. The amino acid sequences are searched for conserved, variable, and hypervariable regions that contribute to the development of a protective immune response against the pathogen.

The antigenic properties of the peptide library can be controlled by selecting the pathogen protein sequences that the library is designed from. For example, a library mimicking a particular subtype of pathogen can be designed by selecting only sequences derived from that subtype. In general, the sequences within any one subtype or sub-subtype are more similar to each other than to sequences from other subtypes throughout their genomes. Group M is the main group of viruses in the HIV-1 global pandemic, and it contains multiple subtypes. Group O is the “outlier” group, and group N is a very distinctive form of the virus that is Non-M, Non-O. The HIV-1 subtypes from the M group of HIV-1, for example, are phylogenetically associated groups or clades of HIV-1 sequences, and are labeled A1, A2, B, C, D, F1, F2, G, H, J and K. In addition, recombinant HIV-1 sequences are possible, where the viral genome includes regions of sequence from more than one subtype. See, for example, the HIV Sequence Database, at hiv-web.lanl.gov. These subtypes represent different lineages of HIV, and have some geographical associations. A library mimicking a particular subtype or recombinant form can be useful, for example, in cases where particular subtypes are found predominantly in particular geographic locations.

The number of potential evolutionary variants of the sequence of an antigenically active region can be enormous. For example, the potential antigenic variability of the V3 region of HIV-1 gp120 is as high as 10²⁵-10³⁰ variants. Once the complete variability at each position in the peptide is determined from the naturally occurring sequences, the variability of the desired VCPL can be reduced by recognizing the most significant amino acids that determine the genotypic diversity of the antigenic variants. The rates of occurrence of various amino acids at variable positions of an immunogenic epitope are determined with regard to their relative occurrence in the alignment of the sequences of the region at issue.

The size of the VCPL can be decreased by recognizing the most antigenically significant amino acids at each variable position, and combining them in groups of variable composition with one or more amino acids representing the groups of variable composition. The amino acids representing groups of variable composition are determined on the basis of antigenic similarity.

Antigenic similarity is a quantitative measure of how readily one amino acid residue can substitute for another in a protein-protein interaction, such as, for example, an antibody-antigen interaction. Two residues are very antigenically similar if substituting one for the other in a particular epitope has a small effect on the antigenic behavior of that epitope. Conversely, if the substitution has a large effect, then the two residues have a low antigenic similarity. In general, residues that have similar physical and chemical characteristics will have a high antigenic similarity.

Antigenic similarity can be expressed in a matrix, called an antigenic similarity matrix (ASM). The construction of a matrix is described in, for example, Maksyutov, A. Z., Eroshkin, A. M., and Kulichkov, V. A, Mol. Biol. 1987, 21:30-47, which is incorporated by reference in its entirety. The matrix can account for the affinity of two residues, that is, how frequently two residues are in contact in a globular protein, as determined by examination of 3D structures. See, for example, Warme, P. K., and Morgan, R. S., J. Mol. Biol. 1978, 118: 289-304, which is incorporated by reference in its entirety. The matrix is also adjusted for the frequency with which an amino acid occurs in the hypervariable regions of immunoglobulins. See, for example, Bourgarit, J., J. Ann. Immunol. 1980, 131D: 267-287, which is incorporated by reference in its entirety.

The values of an initial matrix can be transformed by the formula:
ASMᵢⱼ=100−(Rᵢⱼ+Rⱼᵢ)/2
for each pair of amino acids i and j, and Rᵢⱼ and Rⱼᵢ are elements of the initial matrix, thus producing a symmetric matrix. To differentiate the effects of coinciding amino acids and increase the weight of the residues that occur frequently in antigenic epitopes, the diagonal elements can be adjusted according to the following formula:
ASMᵢᵢ=ASMᵢᵢ+5(hᵢ+sᵢ+c)
where hᵢ is the hydrophilicity of the amino acid (Hopp, T. P., and Woods, K. R., Proc. Natl. Acad Sci. USA 1981, 78:3824-3828, which is incorporated by reference in its entirety), sᵢ is the relative frequency of the amino acid in antigenic determinants of proteins (Maksyutov, A. Z. and Zagrebelnaya, E. S., Comp. Applic. Biosci. 1993, 9:291-297, which is incorporated by reference in its entirety), and c is 6.6, the minimum value of hᵢ+sᵢ, introduced to insure a positive sum (hᵢ+sᵢ+c). Finally, to satisfy the local alignment program SIM, the quantity 75 is subtracted from each element in the matrix. One such matrix is presented in Table 1. The ASM of Table 1 is aimed at the search for locally similar regions exhibiting close structural features in respect to interaction between an antibody or another binding ligand and the binding site (see, for example, Maksyutov A. Z. et al., Molecular Biology 2002, 36: 346-358, which is incorporated by reference in its entirety).

Qualitatively, a matrix value for a pair of amino acids indicates that they are good substitutes for one another, and a low number indicates that they are poor substitutes. For example, valine and leucine have a high antigenic similarity according to the matrix (matrix value of 9), but valine and aspartic acid do not (matrix value of −59), as would be expected from chemical considerations.

TABLE 1

| | A | C | D | E | F | G | H | I | K | L | M | N | P | Q | R | S | T | V | W | Y |
|---|---|---|---|---|---|---|---|---|---|---|---|---|---|---|---|---|---|---|---|---|
| A | 59 | | | | | | | | | | | | | | | | | | | |
| C | −15 | 50 | | | | | | | | | | | | | | | | | | |
| D | −41 | −50 | 81 | | | | | | | | | | | | | | | | | |
| E | −49 | −53 | 4 | 79 | | | | | | | | | | | | | | | | |
| F | −7 | −12 | −49 | −51 | 47 | | | | | | | | | | | | | | | |
| G | 5 | −15 | −25 | −35 | −25 | 49 | | | | | | | | | | | | | | |
| H | −30 | −34 | −42 | −49 | −34 | −35 | 61 | | | | | | | | | | | | | |
| I | 0 | −22 | −62 | −65 | −4 | −35 | −48 | 37 | | | | | | | | | | | | |
| K | −38 | −49 | −48 | −62 | −46 | −35 | −12 | −55 | 88 | | | | | | | | | | | |
| L | 3 | −12 | −55 | −60 | 4 | −30 | −41 | 11 | −47 | 48 | | | | | | | | | | |
| M | −7 | −15 | −57 | −55 | −5 | −30 | −33 | −3 | −46 | 4 | 37 | | | | | | | | | |
| N | −36 | −37 | −20 | −27 | −44 | −15 | −23 | −57 | −23 | −46 | −43 | 55 | | | | | | | | |
| P | −11 | −13 | −28 | −36 | −17 | −15 | −8 | −29 | −24 | −19 | −19 | −19 | 59 | | | | | | | |
| Q | −24 | −24 | −15 | −29 | −29 | −10 | −11 | −43 | −31 | −34 | −30 | 3 | −10 | 71 | | | | | | |
| R | −34 | −42 | −44 | −54 | −39 | −25 | −13 | −51 | 7 | −46 | −45 | −23 | −24 | −26 | 69 | | | | | |
| S | −23 | −31 | −15 | −35 | −34 | −5 | −3 | −45 | −19 | −38 | −40 | −10 | −4 | −5 | −15 | 61 | | | | |
| T | −27 | −30 | −20 | −26 | −33 | −10 | −18 | −46 | −31 | −41 | −42 | −15 | −13 | −11 | −18 | 6 | 62 | | | |
| V | −1 | −16 | −59 | −62 | −4 | −30 | −44 | 13 | −55 | 9 | −1 | −51 | −26 | −37 | −50 | −42 | −39 | 47 | | |
| W | −22 | −21 | −38 | −39 | −18 | −40 | −31 | −27 | −44 | −19 | −22 | −23 | −9 | −19 | −38 | −23 | −17 | −19 | 24 | |
| Y | −25 | −30 | −26 | −20 | −20 | −25 | −25 | −37 | −31 | −27 | −23 | −10 | −13 | −12 | −32 | −19 | −18 | −32 | −8 | 53 |

At each position in the peptide, the matrix value for each pair of residues occurring naturally is looked up. A pair having a positive matrix value will be represented in the library by the residue that occurs more frequently in the natural sequences, and its frequency in the library will be the sum of frequencies for each residue that it represents. The remaining residues are combined in subgroups, and for each subgroup, the matrix is searched for a residue that has a positive matrix value with each residue in the subgroup. If such a residue is found, it is assigned a frequency equal to the sum of frequencies of residues in the subgroup. This residue can be one that does not naturally occur in that position. If a naturally occurring residue does not have a positive matrix value with any other naturally occurring residue at that position, and cannot be represented by another residue not naturally occurring at that position, it will represent itself in the library.

As an example of how the matrix can be used to choose residues for a particular 15 position in a peptide library, consider a variable position in where the following six different residues occur naturally: A (5%), D (20%), E (35%), M (22%), V (10%), and Y (8%). The matrix value for each pair of naturally occurring residues is then looked up:

| | |
|---|---|
| AD | −41 |
| AE | −49 |
| AM | −7 |
| AV | −1 |
| AY | −25 |
| DE | 4 |
| DM | −7 |
| DV | −59 |
| DY | −26 |
| EM | −55 |
| EV | −62 |
| EY | −20 |
| MV | −1 |
| MY | −10 |
| VY | −32 |

Only one pair, DE, has a positive matrix value. That pair of residues will be represented by the more frequent naturally occurring residue, E, and will have a frequency equal to the sum of the frequency of the residues it represents (20+35=55%). The remaining residues, A, M, V and Y, are divided into subgroups and the matrix is searched for other residues that are antigenically similar to these. The subgroup of residues A, M, and V each have a positive matrix value with L:

| | |
|---|---|
| AL | 3 |
| ML | 4 |
| VL | 9 |

Thus, in the library, A, M, and V will be represented by L, which is antigenically similar to all three. The frequency of L in the library is the sum of the frequencies of A, M and V: 5+10+22=37%. Finally, since Y is not antigenically similar to another residue, Y represents itself in the library with a frequency of 8%. Table 2 summarizes how the residues from natural sequences are represented in the library for this example.

TABLE 2

| | |
|---|---|
| Naturally occurring | Library |

| | | | |
|---|---|---|---|
| D | 20% | E | 55% |
| E | 35% | | |
| A | 5% | L | 37% |
| M | 22% | | |
| V | 10% | | |
| Y | 8% | Y | 8% |

Theoretical analysis of the potential cross-reactivity of antibodies raised against peptide libraries shows that in order to induce an immune cross-response sufficiently broad to cover the whole set of antigenic variants of a variable infectious agent would require a library containing tens of thousands of homologous peptides. A smaller library having hundreds of homologous peptides could not induce a sufficiently broad immune response to existing and potential variants of a variable infectious agent. However, increasing the size of the library also increases the danger of that the library will elicit an autoimmune response. The analysis was carried out by methods for estimation of the cross-reactivity of antibodies against homologous antigens. See, for example, Maksyutov A. Z., Voprosy Virusologii 1989, 3: 283-288; Maksyutov A. Z., Eroshkin A. M., and Kulichkov V. A., Molekularnaya Biologiya 1987, 21: 3947; Maksyutov, A. Z., and Zagrebelnaya, E. S., Computer Applications in the Biosciences 1993, 9: 291-297; and Maksyutov, A. Z., and Zagrebelnyi S. N., Molekularnaya Biologiya 1993, 27: 980-991, each of which is incorporated by reference in its entirety.

Typically during the natural infection of an individual and selection of antigenic variants of an infectious agent there is too little time for formation of a sufficient amount of immune effectors capable of cross-reaction with normal human proteins to induce an autoimmune response. Induction of autoimmune processes usually takes a long time, and often requires the constant presence of the sensitizing antigen. Hence, the probability of autosensitization in an individual as a result of local similarity between variants of the hypervariable regions of the variable infectious agent and a human protein may be insignificant. On the other hand, the risk of induction of autoimmune conditions is increased by repeated use of a peptide vaccine for prophylaxis or therapy of an infectious disease. Therefore, during the design of polyepitope vaccine constructs, it should be taken into account that the long persistence of “time-conservative” variants of variable regions of the variable infectious agent can bring about autoimmune conditions, particularly with a vaccine construct containing adjuvants for increasing the immune response. Thus, the design of vaccine constructs on the basis of VCPLs requires comprehensive theoretical consideration of the local similarity between VCPL peptides and the whole set of human proteins.

It can be advantageous to screen the sequences in a library for their potential to generate an autoimmune response. If a peptide in the library is highly similar to a peptide present in a human protein, an immune response could be generated against the human protein, with deleterious health effects for the person. A measure of the likelihood that a peptide sequence might generate an autoimmune response can be calculated by determining the antigenic similarity of the peptide sequence in the library to sequences found in human proteins.

At the final stage of the method of designing a VCPL, the local similarity between the VCPL peptides and the whole set of human proteins can be analyzed. Candidate vaccines on the based on VCPLs can be screened for potentially autoimmunogenic determinants resulting from local similarity of VCPL peptides to human proteins. These potentially autoimmunogenic determinants can be removed from the library.

The epitopes to be modeled in a VCPL can be chosen so as not to be autoimmunogenic. The method of identifying potentially immunopathogenic regions in proteins of an infectious agent on the basis of recognition of regions displaying close local similarity to human proteins provides a key to rational design of safe polyepitope vaccines against pathogenic microorganisms, because it allows elimination of epitopes with an immunopathogenic potential. In particular, antigenic determinants displaying similarity to human proteins that occur often and/or support important physiological functions of the body should be eliminated.

To search for local similarity between a library sequence and human proteins, the SIM program can be used (Huang, X. and Miller, W., Adv. Appl. Math., 1991, 12, 337-357, which is incorporated by reference in its entirety). The sequences of human proteins are available in public databases, for instance SWISS-PROT (rl. 41, FEB-2003) and SP-TrEMBL (rl. 23, MAR-2003). The comparison can be carried out using the version of SIM available on the European Molecular Biology Laboratory (EMBL) server. The following parameters of the SIM program can be used: gap-open penalty 100, gap-extension penalty 20.

The local similarity search may produce a large number of hits, many of which have a small degree of similarity between a peptide sequence from the library and a region of a human protein. It can be useful to filter the results of the similarity search to identify most relevant peptide-protein similarities. The filtering can be performed as follows. The twenty commonly occurring amino acids are divided into intersecting groups with physicochemical similarities: group 1-Lys, Arg; group 2-Asp, Glu; group 3-Asn, Gln; group 4-Ser, Thr; group 5-Ala, Ile, Leu, Val; group 6-Ala, Gly; group 7-Leu, Phe; group 8-Leu, Met, Val; and group 9-Phe, Trp, Tyr.

For each individual hit of local similarity between a library peptide and a human protein, the following values are calculated: index of similarity (Score determined by the SIM program); the percentage of matching amino acids (Match %); the number of matching amino acids (M); the number of mismatching amino acids (MM); the number of gaps (Gap); the number of conservative substitutions (C_{cons}) (that is, the number of substitutions within groups 1, 2, 3 & 4, 5-9); the number of substitutions of charged amino acids (His, Lys, Arg, Asp, Glu) by one of opposite charge (C₊₋); the number of non-conservative substitutions or deletions of charged amino acids (C_{±}); the number of non-conservative substitutions or deletions of polar amino acids (Asn, Gln, Ser, Thr) (Cₚₒₗₐᵣ); and the number of non-conservative substitutions or deletions of aromatic amino acids (Phe, Trp, Tyr) (Cₐᵣₒₘ).

Then an empirical algorithm tests if the following conditions are met:
1) Index of similarity not less than 320 (Score≧320).
2) Number of matching amino acids in a given region not less than 6 (M≧6).
3) Percent of matches not less than 50% (Match % ≧50%).
4) Number of matching amino acids and conservative substitutions in a given region not less than 8: M+C_{cons}≧8. However, if within a homologous region there is a subregion which comprises a string of 8 amino acids in a row, which are either conservative or matching then this region is taken as a whole regardless of the percent of match (Match %).
5) (MM−C_{cons}+Gap)/(M+C_{cons})≦{0.37|_{m=6+∞}}
6) 1.5*C₊₋+C_{±}+0.5*(Cₚₒₗₐᵣ+Cₐᵣₒₘ)≦0.1+0.5*(M+C_{cons}−6)

If all the above criteria are met, then the nature and/or function of the human protein can be considered. If the library sequence is similar to a sequence that occurs frequently in human proteins, or is similar to a sequence in a protein having an important physiological function, that sequence can be removed from the library.

In certain circumstances, it can be desirable for the library to have a small number of sequences, such as fewer than 1,000 sequences, fewer than 500 sequences, or fewer than 250 sequences. The library is representative of the full antigenic diversity found in the naturally occurring sequences. Such a representative chimeric peptide library (RCPL) can be created by selecting sequences from a large library (e.g., a VCPL) such that the selected sequences are representative of the full antigenic diversity found in the naturally occurring sequences and non-natural sequences generated from the large library. The non-natural sequences can reflect potential sequences, for example, by including in a single sequence variants at different positions that have not been found together naturally.

Because an RCPL has a smaller number of peptides than a VCPL, it is inherently less likely to induce an autoimmune response. The sequences chosen for the RCPL can be biased towards certain residues at certain positions, analogous to the residue frequencies in a VCPL. An RCPL can also be biased toward particular discrete sequences. In other words, if an RCPL includes 100 distinct peptide sequences, those peptides can be combined in any arbitrary ratio. Despite the much smaller number of sequences, the antigenic diversity of an RCPL can be as great as that of VCPL from which the RCPL is created. In this way, a mixture of a relatively small number of chemical species (i.e. peptide sequences) can produce an equally broad immune response as a mixture of a very large number of chemical species.

RCPLs are sets of homologous peptides evenly covering the diverse variants of hypervariable regions of infectious agents. RCPLs are characterized by smaller numbers of peptides in the libraries (compared to VCPLs), which evenly cover the set of antigenic variants of hypervariable regions of variable infectious agents, including potential variants. RCPLs are designed to mimic the antigenic diversity of main variable protective determinants of an infectious agent. RCPLs allow production of a wide range of antibodies and cytotoxic T-lymphocytes (CTLs), whose combined specificity covers the set of antigenic variants of the variable infectious agent. Thus, RCPLs can induce a humoral and cellular response against the diversity of current and potential variants of variable infectious agents.

To select the specified number, N, of peptides in the RCPL, a large number (e.g. >1,000, >10,000, >100,000, >1,000,000, or >10,000,000) of peptides sequences are chosen at random from the whole diversity of variants of the mimicked hypervariable antigenically active region (e.g., a VCPL), accounting for the rates of occurrence of amino acids at particular positions of the mimicked region. The set of randomly chosen peptide sequences is arranged in the order of the relative rates of their occurrence in the whole diversity of the variants of the mimicked hypervariable antigenic region. The first 37-100% of the sequences from the set of randomly chosen peptides are used. The percentage used is determined by empirical considerations.

The peptide sequence having amino acids with the greatest rates of occurrence at each position is taken as the first sequence of the RCPL. Alternatively, any peptide of the set of variants of the mimicked hypervariable antigenic region may be used as the first, or reference, sequence.

Each subsequent peptide sequence added to the RCPL is chosen from the set of randomly chosen peptides such that the distance between the new sequence and all sequences previously added to the library is maximal or approaches the maximum. The distance between two peptides, Rᵢⱼ, can be determined with the use of the antigenic similarity matrix (see, e.g., Table 1). Rᵢⱼ is calculated according to:

$${R}_{ij} = {\sum }_{k = 1}^{l} F ( {i}_{k} , {j}_{k} )$$

where iₖ is the k^{th} residue of peptide i, jₖ is the k^{th} residue of peptide j, and l is the number of amino acid residues in each peptide of the library. The function F can be described by an antigenic similarity matrix:
F(iₖ,jₖ)=ASM(iₖ,jₖ)
where the value of ASM(iₖ,jₖ) is the matrix value for the pair of residues represented by iₖ and jₖ.

For each peptide n from the randomly chosen peptides, the sum of distances Sₙ between the first N peptides selected (i.e., those already in the RCPL) and peptide n is calculated according to the formula:

$${S}_{n} = {\sum }_{i = 1}^{N} {R}_{i n}$$

where Rᵢₙ is calculated as desibed above. The peptide n that has the greatest value of Sₙ is added to the library. The selection of sequences from the large set of randomly chosen sequences continues until the RCPL has the desired number of sequences.

The function F can alternatively be used simply to count the number of substitutions between two peptides. In this case, the value of F(iₖ,jₖ) is 1 when iₖ equals jₖ (i.e. both peptides have the same residue at position k), and F(iₖ, jₖ) is 0 when iₖ and jₖ are different. When selected in this way, the sequences of the RCPL should evenly cover the whole set of variants of the mimicked hypervariable antigenically active region.

Each peptide included in the RCPL can be screened for autoimmunogenic properties as described above. If a peptide sequence is determined to have the potential to induce an autoimmune response, it can be excluded from the RCPL. Another candidate, virtually identical in the required properties, is instead chosen as the next peptide in the RCPL. The choice algorithm is designed so that the each new peptide added to the RCPL is chosen from a number of sequences of equal value (i.e. of equal distance from the previous peptide sequence in the library).

A sufficient number of peptides chosen as described here can evenly cover the whole diversity of the antigenic variants of hypervariable epitopes of variable infectious agents. The greater is the number of peptides making up an RCPL, the “denser” the immune response that it can induce. Because the algorithm of choosing N peptides is incremental, the first N/2 peptides also evenly cover the whole diversity of the variants, although less densely.

Libraries of representative chimeric peptides can be constructed as candidate vaccines. Taken together, the peptide sequences in the library cover the whole diversity of variants of hypervariable antigenically active regions of a variable infectious agent. The sequences are designed to mimic the genetic diversity of main variable protective determinants of infectious agents. Also, the mimicking peptides can be chimeric, because, although they mimic the heterogeneous set of variants, the sequences in the RCPL actually occur in few, if any, of the known and potential variants of epitope sequences.

In general, peptide libraries can be synthesized manually or by using an automated synthesizer as described, e.g., in Lebl M. and Krchnak V. (1997) Solid-Phase Peptide Synthesis, Methods in Enzymology, 289, Academic Press, Minneapolis, Minn., which is incorporated by reference in its entirety. The synthesis can be a combinatorial synthesis, in which mixtures of amino acid are coupled to the growing peptide, and a mixture of peptide sequences is formed. In a combinatorial synthesis, amino acids are added to a coupling reaction in proportions dictated by the desired ratio of residues at each position in the peptide, adjusted for the relative coupling rate for each amino acid. Alternatively, the library can be produced by parallel synthesis, in which each sequence is synthesized separately, and the library can be a group of individually pure peptide sequences. Such a library can be converted to a mixture by mixing the pure peptides.

Automated combinatorial synthesis can be preferable when a large library is to be produced. Split synthesis can be preferable when a small library of individual sequences (e.g. an RCPL) is to be produced.

The peptide libraries can be part of a multiple antigenic peptide (MAP). A multiple antigenic peptide includes a branched core from which several peptides extend. The branched core can be based on branching repeats of lysine. The MAP can include for example 4 or 8 peptide chains. The MAP can include a hydrophobic group, such as an alkyl chain derived from palmitic acid. The hydrophobic group can facilitate assembly of the MAP with liposomes or virus-like particles.

The peptides can include the 20 common naturally occurring L-amino acids. The peptides can optionally include corresponding D-amino acids, or mixtures of D and L-amino acids. Non-natural amino acids can likewise be included in the peptides.

The peptide library can be produced by recombinant methods. A DNA construct encoding the peptides of the library can be introduced into a host organism capable of protein expression from the construct. Suitable host organisms can include bacteria such as E. coli, Lactobacillus, and Bifida, and plants, such as tobacco.

The peptide libraries can be used in diagnostic applications, for example to determine the presence of antibodies, or to determine CTL function. For example, the libraries may be used an antigens for an enzyme-linked immunosorbent assay (ELISA) to determine the presence of antibodies in a subject sample. In a solid-phase assay such as an ELISA, the peptides of the library are immobilized on a substrate (for example, in a polystyrene well). The substrate is then exposed to a test sample (e.g., serum from a subject) under conditions that allow specific peptide-antibody complex formation. Any unbound antibodies are then washed away. Antibodies associated with the substrate by virtue of a specific peptide-antibody interaction are then detected. Detection can include exposing the antibodies to a secondary antibody specific for immunoglobulins from the subject. The secondary antibody can be radiolabelled, or coupled to an enzyme such as horseradish peroxidase or alkaline phosphatase, that catalyzes the formation of a colored product.

The diagnostic peptide library can be a broad spectrum diagnostic reagent, or a subtype specific reagent. If the library was designed to be antigenically similar to all known subtypes of a pathogen, then it can be used to detect that a subject has been infected by the pathogen, regardless of what subtype the subject is infected with. If instead the library was designed to be antigenically similar to only one subtype, then it can be used to detect if a subject was infected by the same subtype. A group of subtype specific libraries can be used together to determine which of several subtypes a subject was infected by.

Vaccines based on antigenic peptides are known. See, for example, U.S. Pat. Nos. 4,601,903, 4,599,230, and 4,599,231, each of which is incorporated by reference in its entirety. Vaccines may be prepared as injectables, as liquid solutions or emulsions. The peptides may be mixed with pharmaceutically acceptable excipients that are compatible with the peptides. Excipients may include water, saline, dextrose, glycerol, ethanol, and combinations thereof. The vaccine may further contain auxiliary substances such as wetting or emulsifying agents, pH buffering agents, or adjuvants to enhance the effectiveness of the vaccines. Methods of achieving adjuvant effect for the vaccine include the use of agents such as aluminum hydroxide or phosphate (alum), commonly used as 0.05 to 0.1 percent solution in phosphate buffered saline. Vaccines may be administered parenterally, by injection subcutaneously or intramuscularly. Alternatively, other modes of administration including suppositories and oral formulations may be desirable. For suppositories, binders and carriers may include, for example, polyalkylene glycols or triglycerides. Oral formulations may include normally employed incipients such as, for example, pharmaceutical grades of saccharine, cellulose and magnesium carbonate. These compositions take the form of solutions, suspensions, tablets, pills, capsules, sustained release formulations or powders.

The vaccines are administered in a manner compatible with the dosage formulation, and in such amount as is therapeutically effective, protective and immunogenic. The quantity to be administered depends on the subject to be treated, including, for example, the capacity of the individual's immune system to synthesize antibodies, and to produce a cell-mediated immune response. Precise amounts of active ingredient required to be administered depend on the judgment of the practitioner. However, suitable dosage ranges are readily determinable by one skilled in the art and may be of the order of micrograms of the peptides. Suitable regimes for initial administration and booster doses are also variable, but may include an initial administration followed by subsequent administrations. The dosage of the vaccine may also depend on the route of administration and will vary according to the size of the host.

Nucleic acid molecules encoding the peptides of the library may also be used directly for immunization by administration of the nucleic acid molecules directly, for example by injection, or by first constructing a live vector, such as Salmonella, BCG, adenovirus, poxvirus, vaccinia or poliovirus, and administering the vector.

The vaccine can be a prophylactic vaccine that induces a protective immune response in a subject, before the subject is exposed to the pathogen. In general, a prophylactic vaccine includes a broad-spectrum peptide library. In some cases, a prophylactic vaccine can include a subtype-specific peptide library, for instance, when the subject to be vaccinated is likely to be exposed to only one subtype of the pathogen. The vaccine can be a therapeutic vaccine, designed to induce or enhance an immune response in a subject that is infected by the pathogen. If the subject is known to be infected with a particular subtype of pathogen, the therapeutic vaccine can be one designed to induce an immune response against that subtype.

### EXAMPLES

Peptide libraries were designed based on each of the variable regions V1-V5 of HIV gp120. The sequences used to generate the libraries were from HIV subtype A, B, C, D, F, G, or sequences from all these subtypes were used together. A peptide library generated using sequences from only one subtype can be used as a subtype-specific vaccine, whereas a library generated from multiple subtypes can be used as a broad spectrum vaccine. Each library was initially designed to include fewer than 200,000,000 sequences. From the initial library, smaller libraries were designed to include fewer than 500,000 sequences and fewer than 50,000 sequences. Smaller libraries are designed by removing infrequently occurring residues from the larger library. In Tables 3-37 below, the identities and relative frequencies of residues are given. For example, in Table 3, residue X¹¹ can be S, N or G in the largest library; S or N in the next smaller library, and S in the smallest library. The numbers represent the relative frequencies of each residue at that position. In the largest library, position X¹¹ is occupied by S, N or G in an 80:13:7 ratio; in the next smaller library, X¹¹ is occupied by S or N in an 80:13 ratio; and in the smallest library, every peptide has S at X¹¹.

Chimeric Peptide Libraries—V1 region of HIV gp120

Peptide libraries based on the V1 region of HIV gp120 from subtypes A, B, C, D, F, and G were designed. The libraries had the structure:
C—T—X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—E—X¹⁸—K—N

where X¹-X¹⁸ are defined according to Table 3.

TABLE 3
HIV gp120 V1 region, subtypes ABCDFG

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ |
|---|---|---|---|---|---|---|---|---|---|---|---|---|---|---|---|---|---|---|
| 50,000 | D | L | K | N | T | T | N | T | N | S | S | S | G | M | E | K | G | I |
| | 68 | 81 | 43 | 87 | 33 | 81 | 67 | 69 | 70 | 61 | 80 | 77 | 49 | 59 | 88 | 58 | 79 | 73 |
| | N | | T | | N | | T | | T | N | | N | S | T | | E | E | M |
| | 32 | | 22 | | 26 | | 33 | | 30 | 20 | | 15 | 33 | 15 | | 16 | 21 | 27 |
| | | | N | | A | | | | | | | | N | I | | | | |
| | | | 18 | | 24 | | | | | | | | 18 | 14 | | | | |
| | | | | | D | | | | | | | | | | | | | |
| | | | | | 17 | | | | | | | | | | | | | |
| 500,000 | | | | T | | | | N | | | N | | | | K | | | |
| | | | | 13 | | | | 13 | | | 13 | | | | 12 | | | |
| 200,000,000 | | W | G | | | N | | V | | A | G | E | | E | | G | | |
| | | 12 | 8 | | | 10 | | 11 | | 12 | 7 | 8 | | 12 | | 10 | | |
| | | Y | E | | | V | | A | | I | | | | | | N | | |
| | | 7 | 8 | | | 10 | | 7 | | 7 | | | | | | 9 | | |
| | | | | | | | | | | | | | | | | T | | |
| | | | | | | | | | | | | | | | | 8 | | |

Peptide libraries based on the V1 region of HIV gp120 from subtype A were designed. The libraries had the structure:
C—T—X¹—X²—X³—X⁴—X⁵—X⁶—X⁷—X⁸—X⁹—X¹⁰—X¹¹—X¹²—X¹³—X¹⁴—X¹⁵—X¹⁶—E—X¹⁷—K—N

where X¹-X¹⁷ are defined according to Table 4.

TABLE 4
HIV gp120 V1 region, subtype A

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ |
|---|---|---|---|---|---|---|---|---|---|---|---|---|---|---|---|---|---|
| 50,000 | N | V | N | V | T | S | T | S | N | T | T | E | G | D | K | E | I |
| | 87 | 67 | 70 | 76 | 65 | 53 | 62 | 42 | 45 | 34 | 57 | 32 | 37 | 29 | 62 | 69 | 82 |
| | D | A | T | S | N | N | N | N | A | I | | T | E | M | E | G | |
| | 13 | 33 | 30 | 24 | 24 | 32 | 20 | 36 | 40 | 29 | | 26 | 34 | 21 | 19 | 31 | |
| | | | | | | | | | | | | N | | N | | | |
| | | | | | | | | | | | | 22 | | 20 | | | |
| 500,000 | | | | | | | | | S | N | | | S | | Q | | M |
| | | | | | | | | | 15 | 19 | | | 16 | | 19 | | 18 |
| | | | | | | | | | | D | | | | | | | |
| | | | | | | | | | | 18 | | | | | | | |
| 200,000,000 | | | | | V | V | V | V | | | I | V | N | G | | | |
| | | | | | 11 | 15 | 10 | 14 | | | 13 | 13 | 13 | 14 | | | |
| | | | | | | | A | A | | | R | Y | | T | | | |
| | | | | | | | 8 | 8 | | | 13 | 8 | | 9 | | | |
| | | | | | | | | | | | D | | | I | | | |
| | | | | | | | | | | | 9 | | | 7 | | | |
| | | | | | | | | | | | N | | | | | | |
| | | | | | | | | | | | 7 | | | | | | |

Peptide libraries based on the V1 region of HIV gp120 from subtype B were designed. The libraries had the structure:
C—T—X¹—X²—X³—N—X⁴—X⁵—X⁶—X⁷—X⁸—X⁹—X¹⁰—X¹¹—X¹²—X¹³—X¹⁴—X¹⁵—E—X¹⁶—X¹⁷—E—X¹⁸—K—N

where X¹-X¹⁸ are defined according to Table 5.

TABLE 5
HIV gp120 V1 region, subtype B

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ |
|---|---|---|---|---|---|---|---|---|---|---|---|---|---|---|---|---|---|---|
| 50,000 | D | L | K | A | T | N | T | N | S | S | S | G | E | I | M | K | G | I |
| | 85 | 79 | 55 | 30 | 91 | 74 | 90 | 66 | 74 | 83 | 88 | 49 | 29 | 34 | 67 | 69 | 92 | 75 |
| | | | N | T | | T | | T | N | | | S | M | T | I | G | | M |
| | | | 14 | 30 | | 26 | | 34 | 19 | | | 36 | 20 | 22 | 14 | 12 | | 25 |
| | | | T | N | | | | | | | | N | G | M | | | | |
| | | | 12 | 21 | | | | | | | | 15 | 17 | 18 | | | | |
| | | | | D | | | | | | | | | T | K | | | | |
| | | | | 19 | | | | | | | | | 17 | 18 | | | | |
| 500,000 | N | W | | | | | | | | | N | | | | T | | | |
| | 15 | 14 | | | | | | | | | 12 | | | | 12 | | | |
| 200,000,000 | | Y | E | | N | | N | | A | N | | | I | E | E | E | E | |
| | | 7 | 11 | | 9 | | 10 | | 7 | 8 | | | 9 | 8 | 7 | 11 | 8 | |
| | | | G | | | | | | | G | | | K | | | T | | |
| | | | 9 | | | | | | | 8 | | | 8 | | | 8 | | |

Peptide libraries based on the V1 region of HIV gp120 from subtype C were designed. The libraries had the structure:
C—X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—E—X¹⁸—K—N

where X¹-X¹⁸ are defined according to Table 6.

TABLE 6
HIV gp120 V1 region, subtype C

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ |
|---|---|---|---|---|---|---|---|---|---|---|---|---|---|---|---|---|---|---|
| 50,000 | T | N | A | T | R | N | G | N | V | T | Y | N | N | T | M | E | G | I |
| | 73 | 86 | 48 | 76 | 28 | 63 | 48 | 68 | 43 | 84 | 52 | 67 | 59 | 80 | 58 | 30 | 57 | 61 |
| | | | V | N | S | T | N | T | T | | V | | D | | I | K | E | M |
| | | | 41 | 24 | 24 | 30 | 22 | 32 | 33 | | 21 | | 21 | | 24 | 27 | 34 | 39 |
| | | | | | N | | S | | A | | | | | | | N | | |
| | | | | | 20 | | 22 | | 24 | | | | | | | 26 | | |
| 500,000 | R | | | | V | | | | | | S | | | N | T | | | |
| | 19 | | | | 18 | | | | | | 18 | | | 20 | 18 | | | |
| 200,000,000 | V | D | T | | A | D | V | | | N | N | T | S | | | T | K | |
| | 8 | 14 | 11 | | 10 | 7 | 9 | | | 16 | 9 | 18 | 11 | | | 17 | 9 | |
| | | | | | | | | | | | | D | G | | | | | |
| | | | | | | | | | | | | 15 | 9 | | | | | |

Peptide libraries based on the V1 region of HIV gp120 from subtype D were designed. The libraries had the structure:
C—X¹—X²—X³—X⁴—X⁵—X⁶—X⁷—T—X⁸—X⁹—X¹⁰—X¹¹—X¹²—X¹³—X¹⁴—X¹⁵—X¹⁶—E—X¹⁷—K—N

where X¹-X¹⁷ are defined according to Table 7.

TABLE 7
HIV gp120 V1 region, subtype D

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ |
|---|---|---|---|---|---|---|---|---|---|---|---|---|---|---|---|---|---|
| 50,000 | T | D | W | K | N | N | T | T | N | N | N | T | T | M | E | G | M |
| | 87 | 85 | 38 | 45 | 47 | 73 | 35 | 39 | 44 | 55 | 47 | 45 | 75 | 32 | 64 | 71 | 91 |
| | | | A | W | G | E | A | N | G | S | T | V | D | I | G | | |
| | | | 20 | 18 | 28 | 28 | 29 | 24 | 25 | 29 | 24 | 37 | 25 | 21 | 36 | | |
| | | | | | S | | | | K | | K | | | E | | | |
| | | | | | 25 | | | | 17 | | 18 | | | 19 | | | |
| 500,000 | | | | | | | N | D | | | | A | | | | I | |
| | | | | | | | 18 | 19 | | | | 18 | | | | 17 | |
| | | | | | | | Y | G | | | | | | | | | |
| | | | | | | | 18 | 18 | | | | | | | | | |
| 200,000,000 | I | N | V | T | | | | | T | K | A | | | N | | T | V |
| | 13 | 15 | 17 | 16 | | | | | 14 | 16 | 11 | | | 10 | | 12 | 9 |
| | | | E | N | | | | | | | | | | K | | | |
| | | | 15 | 11 | | | | | | | | | | 9 | | | |
| | | | Y | L | | | | | | | | | | T | | | |
| | | | 11 | 11 | | | | | | | | | | 9 | | | |

Peptide libraries based on the V1 region of HIV gp120 from subtype F were designed. The libraries had the structure:
C—X¹—X²—X³—X⁴—X⁵—X⁶—T—X⁷—X⁸—X⁹—X¹⁰—X¹¹—X¹²—X¹³T—L—K—X¹⁴—X¹⁵—X¹⁶—X¹⁷—E—X¹⁸—X¹⁹—X²⁰—N

where X¹-X²⁰ are defined according to Table 8.

TABLE 8
HIV gp120 V1 region, subtype F

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ | X²⁰ |
|---|---|---|---|---|---|---|---|---|---|---|---|---|---|---|---|---|---|---|---|---|
| 50,000 | T | N | A | T | N | A | N | D | T | I | T | N | T | E | E | P | G | A | I | Q |
| | 60 | 47 | 37 | 53 | 61 | 84 | 73 | 40 | 60 | 54 | 37 | 53 | 56 | 90 | 89 | 70 | 80 | 65 | 86 | 67 |
| | R | D | T | I | T | | S | N | N | T | N | G | D | | | | | E | | K |
| | 28 | 32 | 25 | 47 | 27 | | 27 | 27 | 27 | 46 | 32 | 35 | 31 | | | | | 35 | | 33 |
| | | | N | | | | | | | | A | | | | | | | | | |
| | | | 25 | | | | | | | | 31 | | | | | | | | | |
| 500,000 | | | | | | T | | G | | | | | | | | Q | E | | | |
| | | | | | | 16 | | 20 | | | | | | | | 18 | 20 | | | |
| 200,000,000 | A | A | I | | V | | | S | A | | | D | P | I | S | S | | | M | |
| | 12 | 11 | 13 | | 12 | | | 13 | 13 | | | 12 | 13 | 10 | 11 | 12 | | | 14 | |
| | | S | | | | | | | | | | | | | | | | | | |
| | | 11 | | | | | | | | | | | | | | | | | | |

Peptide libraries based on the V1 region of HIV gp120 from subtype G were designed. The libraries had the structure:
C—T—X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—N

where X¹-X¹⁷ are defined according to Table 9.

TABLE 9
HIV gp120 V1 region, subtype G

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ |
|---|---|---|---|---|---|---|---|---|---|---|---|---|---|---|---|---|---|
| 50,000 | N | V | T | N | N | S | T | K | N | V | T | E | K | E | E | I | K |
| | 77 | 88 | 65 | 67 | 71 | 34 | 70 | 35 | 72 | 62 | 56 | 51 | 44 | 53 | 52 | 73 | 78 |
| | D | | N | | T | C | N | T | | S | E | S | E | N | R | M | |
| | 23 | | 35 | | 29 | 26 | 16 | 25 | | 22 | 17 | 19 | 24 | 31 | 29 | 27 | |
| | | | | | | N | | N | | | | | | A | N | | |
| | | | | | | 24 | | 16 | | | | | | 17 | 19 | | |
| 500,000 | | | | | | G | | | | C | C | G | G | | | | |
| | | | | | | 16 | | | | 16 | 15 | 16 | 17 | | | | |
| | | | | | | | | | | | | N | N | | | | |
| | | | | | | | | | | | | 14 | 15 | | | | |
| 200,000,000 | | A | | Y | | | A | E | E | | V | | | | | | T |
| | | 12 | | 12 | | | 14 | 14 | 14 | | 11 | | | | | | 12 |
| | | | | K | | | | G | T | | | | | | | | E |
| | | | | 10 | | | | 10 | 14 | | | | | | | | 10 |
| | | | | T | | | | | | | | | | | | | |
| | | | | 10 | | | | | | | | | | | | | |

Chimeric Peptide Libraries—V2 region of HIV gp120

Peptide libraries based on the V2 region of HIV gp120 from subtypes A, B, C, D, F, and G were designed. The libraries had the structure:
S—X¹—N—X²—T—T—X³—X⁴—R—X⁵—K—X⁶—X⁷—X⁸—X⁹—X¹⁰—X¹¹—L—F—Y—X¹²—L—D—V—V—X¹³—I—X¹⁴—X¹⁵—X¹⁶—X¹⁷—X¹⁸—X¹⁹—Y—X²⁰—L—X²¹—X²²—C

where X¹-X²² are defined according to Table 10.

TABLE 10
HIV gp120 V2 region, subtypes A, B, C, D, F, G

| Library size < | X¹ | X² | X³ | X⁴ | X⁵ | X⁶ | X⁷ | X⁸ | X⁹ | X¹⁰ | X¹¹ |
|---|---|---|---|---|---|---|---|---|---|---|---|
| 50,000 | F | I | E | I | D | V | Q | K | E | Y | A |
| | 91 | 84 | 37 | 91 | 95 | 45 | 69 | 89 | 62 | 89 | 79 |
| | | M | S | | | K | | | V | | S |
| | | 16 | 32 | | | 40 | | | 33 | | 21 |
| | | | N | | | M | | | | | |
| | | | 18 | | | 15 | | | | | |
| 500,000 | | | | M | | | K | Q | | H | |
| | | | | 9 | | | 31 | 11 | | 11 | |
| 200,000,000 | Y | | V | | N | | | | Q | | |
| | 9 | | 7 | | 5 | | | | 5 | | |
| | | | G | | | | | | | | |
| | | | 6 | | | | | | | | |

| Library size < | X¹² | X¹³ | X¹⁴ | X¹⁵ | X¹⁶ | X¹⁷ | X¹⁸ | X¹⁹ | X²⁰ | X²¹ | X²² |
|---|---|---|---|---|---|---|---|---|---|---|---|
| 50,000 | K | P | D | N | D | S | T | S | R | I | S |
| | 93 | 73 | 63 | 78 | 54 | 51 | 91 | 76 | 95 | 95 | 51 |
| | | Q | N | D | N | N | | N | | | N |
| | | 27 | 22 | 22 | 32 | 49 | | 10 | | | 49 |
| | | | | | S | | | | | | |
| | | | | | 15 | | | | | | |
| 500,000 | | | | | | | N | | | | |
| | | | | | | | 9 | | | | |
| 200,000,000 | S | | G | | | | | E | T | T | |
| | 7 | | 8 | | | | | 8 | 5 | 5 | |
| | | | K | | | | | R | | | |
| | | | 7 | | | | | 6 | | | |

Peptide libraries based on the V2 region of HIV gp120 from subtype A were designed. The libraries had the structure:
S—X¹—X²—X³—T—T—X⁴—L—R—D—K—K—X⁵—X⁶—V—X⁷—X⁸—L—F—Y—R—L—D—V—V—X⁹—I—X¹⁰—X¹¹—X¹²—X¹³—X¹⁴—X¹⁵—X¹⁶—X¹⁷—X¹⁸—Y—R—L—I—X¹⁹—C

where X¹-X¹⁹ are defined according to Table 11.

TABLE 11
HIV gp120 V2 region, subtype A

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ |
|---|---|---|---|---|---|---|---|---|---|---|---|---|---|---|---|---|---|---|---|
| 50,000 | F | N | M | E | Q | K | Y | S | Q | N | E | N | N | S | S | N | S | E | N |
| | 63 | 91 | 58 | 92 | 62 | 90 | 79 | 83 | 59 | 61 | 68 | 62 | 71 | 86 | 82 | 69 | 59 | 60 | 96 |
| | Y | | I | | K | | H | A | P | D | | S | T | | N | S | N | Q | |
| | 37 | | 42 | | 38 | | 15 | 17 | 35 | 20 | | 15 | 20 | | 18 | 18 | 32 | 18 | |
| | | | | | | | | | | K | | G | | | | D | | S | |
| | | | | | | | | | | 13 | | 10 | | | | 13 | | 16 | |
| 500,000 | | | | | | Q | | | | | Q | | D | | | | | | |
| | | | | | | 10 | | | | | 11 | | 9 | | | | | | |
| | | | | | | | | | | | A | | | | | | | | |
| | | | | | | | | | | | 11 | | | | | | | | |
| | | | | | | | | | | | K | | | | | | | | |
| | | | | | | | | | | | 9 | | | | | | | | |
| 200,000,000 | | S | | V | | | S | | K | T | | K | | N | | | E | K | S |
| | | 9 | | 8 | | | 6 | | 6 | 6 | | 8 | | 9 | | | 4 | 6 | 4 |
| | | | | | | | | | | | | D | | A | | | K | | |
| | | | | | | | | | | | | 4 | | 5 | | | 4 | | |

Peptide libraries based on the V2 region of HIV gp120 from subtype B were designed. The libraries had the structure:
S—F—X¹—I—T—T—X²—X³—X⁴—X⁵—K—X⁶—X⁷—X⁸—X⁹—X¹⁰—A—X¹¹—F—X¹²—X¹³—L—D—V—V—X¹⁴—I—X¹⁵—X¹⁶—X¹⁷—X¹⁸—T—X¹⁹—Y—X²⁰—L—X²¹—X²²—C

where X¹-X²² are defined according to Table 12.

TABLE 12
HIV gp120 V2 region, subtype B

| Library size < | X¹ | X² | X³ | X⁴ | X⁵ | X⁶ | X⁷ | X⁸ | X⁹ | X¹⁰ | X¹¹ |
|---|---|---|---|---|---|---|---|---|---|---|---|
| 50,000 | N | S | I | R | D | V | Q | K | E | Y | L |
| | 94 | 53 | 85 | 94 | 85 | 68 | 80 | 95 | 93 | 95 | 95 |
| | | N | M | | N | M | K | | | | |
| | | 28 | 15 | | 8 | 24 | 20 | | | | |
| | | G | | | | R | | | | | |
| | | 11 | | | | 8 | | | | | |
| | | D | | | | | | | | | |
| | | 9 | | | | | | | | | |
| 500,000 | | | | | | | | | Q | | |
| | | | | | | | | | 7 | | |
| 200,000,000 | K | | | S | G | | | E | | H | T |
| | 6 | | | 6 | 7 | | | 5 | | 5 | 5 |

| Library size < | X¹² | X¹³ | X¹⁴ | X¹⁵ | X¹⁶ | X¹⁷ | X¹⁸ | X¹⁹ | X²⁰ | X²¹ | X²² |
|---|---|---|---|---|---|---|---|---|---|---|---|
| 50,000 | Y | K | P | D | N | D | N | S | R | I | S |
| | 95 | 91 | 88 | 79 | 76 | 70 | 61 | 87 | 92 | 86 | 81 |
| | | S | Q | G | D | N | T | | T | T | N |
| | | 9 | 13 | 11 | 18 | 22 | 39 | | 8 | 7 | 19 |
| 500,000 | | | | | | S | | R | | R | |
| | | | | | | 7 | | 7 | | 7 | |
| 200,000,000 | N | | | K | K | | | N | | | |
| | 5 | | | 5 | 7 | | | 6 | | | |
| | | | | N | | | | | | | |
| | | | | 5 | | | | | | | |

Peptide libraries based on the V2 region of HIV gp120 from subtype C were designed. The libraries had the structure:
S—F—N—X¹—T—T—E—L—R—D—K—X²—X³—X⁴—X⁵—X⁶—A—L—F—Y—R—X⁷—D—I—V—X⁸—L—X⁹—X¹⁰—X¹¹—X¹²—X¹³—X¹⁴—Y—X¹⁵—L—I—X¹⁶—C

where X¹-X¹⁶ are defined according to Table 13.

TABLE 13
HIV gp 120 V2 region, subtype C

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ |
|---|---|---|---|---|---|---|---|---|---|---|---|---|---|---|---|---|
| 50,000 | I | K | K | K | V | Y | L | P | N | N | N | S | S | E | R | N |
| | 45 | 88 | 49 | 61 | 53 | 60 | 94 | 87 | 63 | 71 | 56 | 89 | 71 | 76 | 90 | 96 |
| | A | | Q | Q | E | H | | Q | D | S | S | | G | T | I | |
| | 21 | | 42 | 26 | 35 | 23 | | 8 | 28 | 18 | 32 | | 14 | 10 | 10 | |
| | T | | | T | A | | | | K | | | | R | | | |
| | 17 | | | 13 | 11 | | | | 9 | | | | 9 | | | |
| | M | | | | | | | | | | | | | | | |
| | 16 | | | | | | | | | | | | | | | |
| 500,000 | | T | | | | | | | | D | | G | | K | | |
| | | 7 | | | | | | | | 7 | | 7 | | 7 | | |
| 200,000,000 | | Q | E | | | N | P | S | | K | D | N | N | N | | S |
| | | 5 | 4 | | | 6 | 6 | 5 | | 4 | 7 | 4 | 5 | 6 | | 4 |
| | | | H | | | S | | | | | A | | | | | |
| | | | 4 | | | 5 | | | | | 5 | | | | | |
| | | | | | | R | | | | | | | | | | |
| | | | | | | 5 | | | | | | | | | | |

Peptide libraries based on the V2 region of HIV gp120 from subtype D were designed. The libraries had the structure:
S—F—N—I—T—T—X¹—V—X²—D—K—X³—X⁴—X⁵—X⁶—X⁷—A—L—X⁸—Y—R—X⁹—D—V—V—X¹⁰—X¹¹—X¹²—X¹³—X¹⁴—X¹⁵—X¹⁶—X¹⁷—X¹⁸—Y—R—L—I—X¹⁹—C

where X¹-X¹⁹ are defined according to Table 14.

TABLE 14
HIV gp120 V2 region subtype D

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ |
|---|---|---|---|---|---|---|---|---|---|---|---|---|---|---|---|---|---|---|---|
| 50,000 | V | R | K | K | Q | V | Y | F | L | Q | I | D | N | Q | N | S | S | S | N |
| | 58 | 93 | 91 | 86 | 86 | 57 | 50 | 97 | 96 | 77 | 85 | 83 | 79 | 62 | 80 | 83 | 87 | 83 | 96 |
| | E | | Q | | K | E | H | | | P | M | N | T | S | T | Q | A | N | |
| | 38 | | 9 | | 10 | 34 | 35 | | | 23 | 15 | 11 | 12 | 22 | 20 | 13 | 8 | 14 | |
| | | | | | | | Q | | | | | | | K | | | | | |
| | | | | | | | 10 | | | | | | | 13 | | | | | |
| 500,000 | | I | | E | | | | | | | | G | D | | | | | | |
| | | 7 | | 7 | | | | | | | | 6 | 8 | | | | | | |
| 200,000,000 | | | | Q | | | | | | | | | | | | | | | |
| | | | | 7 | | | | | | | | | | | | | | | |
| | A | | | | E | K | S | Y | P | | | | | D | | G | N | D | H |
| | 4 | | | | 4 | 5 | 5 | 3 | 4 | | | | | 3 | | 2 | 5 | 3 | 4 |
| | | | | | | A | | | | | | | | | | K | | | |
| | | | | | | 4 | | | | | | | | | | 2 | | | |

Peptide libraries based on the V2 region of HIV gp120 from subtype F were designed. The libraries had the structure:
S—F—N—X¹—T—X²—E—V—R—D—K—X³—X⁴—K—X⁵—X⁶—A—L—F—Y—R—L—D—I—X⁷—X⁸—I—X⁹—X¹⁰—X¹¹—X¹²—X¹³—X¹⁴—X¹⁵—Y—R—L—X¹⁶—X¹⁷—X¹⁸

where X¹-X¹⁸ are defined according to Table 15.

TABLE 15
HIV gp120 region V2, subtype F

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ |
|---|---|---|---|---|---|---|---|---|---|---|---|---|---|---|---|---|---|---|
| 50,000 | I | T | Q | K | V | H | V | P | N | N | S | S | N | S | T | I | N | C |
| | 50 | 90 | 53 | 47 | 67 | 54 | 85 | 75 | 56 | 72 | 35 | 50 | 73 | 42 | 47 | 95 | 57 | 95 |
| | M | | K | L | E | Q | | Q | S | | N | D | S | R | E | | S | |
| | 30 | | 47 | 29 | 19 | 21 | | 25 | 24 | | 30 | 28 | 27 | 37 | 38 | | 24 | |
| | T | | | Q | | | | | | | D | N | | | | | | |
| | 20 | | | 24 | | | | | | | 20 | 22 | | | | | | |
| 500,000 | | | | | | Y | E | | | | G | | | | I | | H | |
| | | | | | | 15 | 15 | | | | 15 | | | | 15 | | 14 | |
| 200,000,000 | | M | | | Q | S | | | K | K | | | | G | | T | G | I |
| | | 10 | | | 14 | 11 | | | 10 | 14 | | | | 10 | | 5 | 5 | 5 |
| | | | | | | | | | G | A | | | | N | | | | |
| | | | | | | | | | 10 | 7 | | | | 5 | | | | |
| | | | | | | | | | | M | | | | D | | | | |
| | | | | | | | | | | 7 | | | | 5 | | | | |

Peptide libraries based on the V2 region of HIV gp120 from subtype G were designed. The libraries had the structure:
S—F—N—X¹—T—X²—X³—X⁴—X⁵—D—K—X⁶—K—X⁷—E—Y—A—L—F—Y—R—X⁸—D—V—V—X⁹—I—X¹⁰—X¹¹—X¹²—X¹³—X¹⁴—X¹⁵—Y—X¹⁶—L—X¹⁷—X¹⁸—C

where X¹-X¹⁸ are defined according to Table 16.

TABLE 16
HIV gp120 V2 region, subtype G

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ |
|---|---|---|---|---|---|---|---|---|---|---|---|---|---|---|---|---|---|---|
| 50,000 | I | T | E | I | R | K | T | L | P | N | D | N | N | N | N | R | I | N |
| | 94 | 95 | 97 | 97 | 93 | 80 | 51 | 95 | 91 | 60 | 85 | 49 | 54 | 63 | 66 | 87 | 95 | 97 |
| | | | | | Q | Q | K | | | D | G | S | S | S | S | V | | |
| | | | | | 7 | 10 | 29 | | | 28 | 15 | 24 | 27 | 31 | 27 | 10 | | |
| | | | | | | T | A | | | T | | G | D | G | D | | | |
| | | | | | | 7 | 8 | | | 8 | | 13 | 10 | 6 | 7 | | | |
| | | | | | | | E | | | | | D | R | | | | | |
| | | | | | | | 7 | | | | | 9 | 6 | | | | | |
| 500,000 | | | | | | | Q | T | Q | | | | | | | | T | |
| | | | | | | | 5 | 5 | 5 | | | | | | | | 5 | |
| 200,000,000 | M | A | A | M | | E | | | S | R | | K | I | | | T | | H |
| | 3 | 5 | 3 | 3 | | 3 | | | 4 | 4 | | 4 | 3 | | | 3 | | 3 |
| | T | | | | | | | | | | | | | | | | | |
| | 3 | | | | | | | | | | | | | | | | | |

Chimeric Peptide Libraries—V3 Region of HIV gp120

Peptide libraries based on the V3 region of HIV gp120 from subtypes A, B, C, D, F, and G were designed. The libraries had the structure:
X¹C—X²—R—P—X³—N—N—T—R—X⁴—X⁵—X⁶—X⁷—X⁸—G—X⁹—G—X¹⁰—X¹¹—X¹²—X¹³—X¹⁴—T—G—X¹⁵—I—X¹⁶—G—X¹⁷—I—R—X¹⁸—A—X¹⁹—C—X²⁰

where X¹-X²⁰ are defined according to Table 17.

TABLE 17
HIV gp120 V3 region, subtypes A, B, C, D, F, G

| Library size < | X¹ | X² | X³ | X⁴ | X⁵ | X⁶ | X⁷ | X⁸ | X⁹ | X¹⁰ |
|---|---|---|---|---|---|---|---|---|---|---|
| 50,000 | N | T | N | K | S | I | H | I | P | R |
| | 78 | 89 | 77 | 90 | 78 | 92 | 45 | 93 | 95 | 53 |
| | V | I | S | | G | | R | | | Q |
| | 9 | 11 | 10 | | 15 | | 29 | | | 47 |
| | T | | | | | | P | | | |
| | 8 | | | | | | 12 | | | |
| | | | | | | | S | | | |
| | | | | | | | 8 | | | |
| 500,000 | | | G | | R | | N | M | | |
| | | | 8 | | 7 | | 6 | 7 | | |
| 200,000,000 | | | | Q | | T | | | | |
| | | | | 5 | | 4 | | | | |
| | E | | Y | T | | M | | | L | |
| | 4 | | 4 | 4 | | 4 | | | 5 | |

| Library size < | X¹¹ | X¹² | X¹³ | X¹⁴ | X¹⁵ | X¹⁶ | X¹⁷ | X¹⁸ | X¹⁹ | X²⁰ |
|---|---|---|---|---|---|---|---|---|---|---|
| 50,000 | A | F | Y | A | D | 1 | D | Q | H | N |
| | 72 | 90 | 92 | 60 | 68 | 96 | 86 | 85 | 85 | 95 |
| | T | W | | T | Q | | N | K | Y | |
| | 23 | 10 | | 40 | 14 | | 14 | 15 | 15 | |
| | | | | | A | | | | | |
| | | | | | 11 | | | | | |
| 500,000 | | | | | | | | | | |
| 200,000,000 | | | F | | | | | | | |
| | | | 4 | | | | | | | |
| | V | | H | | R | T | | | | T |
| | 4 | | 3 | | 7 | 4 | | | | 5 |

Peptide libraries based on the V3 region of HIV gp120 from subtype A were designed. The libraries had the structure:
X¹—C—X²—R—P—X³—N—N—X⁴—R—X⁵—X⁶—V—X⁷—I—G—P—G—X⁸—X⁹—F—X¹⁰—X¹¹—X¹²—X¹³—X¹⁴—I—X¹⁵—G—X¹⁶—I—R—X¹⁷—A—X¹⁸—C—X¹⁹

where X¹-X¹⁹ are defined according to Table 18. A dash “—” indicates no residue at that position. For example, if X¹² is represented by a dash, then no residue occupies X12, and the residue of X¹¹ is bound directly to the residue of X¹³.

TABLE 18
HIV gp120 V3 region, subtype A

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ |
|---|---|---|---|---|---|---|---|---|---|---|---|---|---|---|---|---|---|---|---|
| 50,000 | N | T | N | T | K | S | R | Q | A | Y | A | T | G | D | I | D | Q | H | N |
| | 76 | 72 | 68 | 97 | 78 | 85 | 66 | 93 | 56 | 95 | 95 | 91 | 89 | 79 | 92 | 84 | 76 | 76 | 87 |
| | T | I | G | | T | G | H | R | T | | | | — | A | T | N | K | Y | T |
| | 19 | 28 | 23 | | 19 | 11 | 34 | 7 | 41 | | | | 6 | 12 | 8 | 16 | 22 | 24 | 7 |
| | | | S | | | | | | | | | | | | | | | | |
| | | | 9 | | | | | | | | | | | | | | | | |
| 500,000 | | | | | | | | | | | T | R | D | | | | | | E |
| | | | | | | | | | | | 5 | 5 | 5 | | | | | | 5 |
| 200,000,000 | D | | | K | Q | R | | | V | F | | A | | N | | | E | | |
| | 5 | | | 3 | 3 | 2 | | | 3 | 3 | | 2 | | 4 | | | 2 | | |
| | | | | | | N | | | | H | | — | | R | | | | | |
| | | | | | | 2 | | | | 2 | | 2 | | 3 | | | | | |
| | | | | | | | | | | | | | | V | | | | | |
| | | | | | | | | | | | | | | 2 | | | | | |

Peptide libraries based on the V3 region of HIV gp120 from subtype B were designed. The libraries had the structure:
X¹—C—X²—R—P—X³—N—N—T—R—K—X⁴—I—X⁵—X⁶—G—X⁷—G—X⁸—X⁹—X¹⁰—X¹¹—X¹²—T—X¹³—X¹⁴—I—X¹⁵—G—X¹⁶—I—R—X¹⁷—A—X¹⁸—C—X¹⁹

where X¹-X¹⁹ are defined according to Table 19.

TABLE 19
HIV gp120 V3 region, subtype B

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ |
|---|---|---|---|---|---|---|---|---|---|---|---|---|---|---|---|---|---|---|---|
| 50,000 | N | T | N | S | H | I | P | R | A | F | Y | T | G | E | I | D | Q | H | N |
| | 94 | 91 | 86 | 75 | 58 | 89 | 94 | 85 | 90 | 80 | 91 | 59 | 94 | 62 | 97 | 88 | 89 | 91 | 95 |
| | | I | S | G | P | M | | Q | | W | | A | | Q | | N | K | Y | T |
| | | 9 | 10 | 18 | 18 | 11 | | 9 | | 15 | | 41 | | 21 | | 13 | 11 | 9 | 5 |
| | | | | R | N | | | | | | | | | R | | | | | |
| | | | | 7 | 11 | | | | | | | | | 9 | | | | | |
| | | | | | T | | | | | | | | | G | | | | | |
| | | | | | 11 | | | | | | | | | 9 | | | | | |
| 500,000 | | | | | | | | | V | | F | | | | | | | | |
| | | | | | | | | | 5 | | 4 | | | | | | | | |
| 200,000,000 | | | | | | | | | T | | H | | | | | | | | |
| | | | | | | | | | 5 | | 4 | | | | | | | | |
| | T | | G | | Y | | L | G | | V | | | E | | T | | | | |
| | 4 | | 4 | | 3 | | 3 | 3 | | 4 | | | 3 | | 3 | | | | |
| | H | | | | | | W | S | | | | | R | | | | | | |
| | 2 | | | | | | 3 | 3 | | | | | 3 | | | | | | |

Peptide libraries based on the V3 region of HIV gp120 from subtype C were designed. The library had the structure:
X¹—C—X²—R—P—X³—N—N—T—R—X⁴—X⁵—X⁶—X⁷—I—G—P—G—Q—X⁸—F—X⁹—X¹⁰—T—X¹¹—X¹²—I—X¹³—G—X¹⁴—I—R—X¹⁵—A—X¹⁶—C—X¹⁷

where X¹-X¹⁷ are defined according to Table 20.

TABLE 20
HIV gp120 V3 region, subtype C

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ |
|---|---|---|---|---|---|---|---|---|---|---|---|---|---|---|---|---|---|
| 50,000 | V | T | N | K | S | I | R | T | Y | A | G | D | I | D | Q | H | N |
| | 57 | 91 | 77 | 88 | 98 | 76 | 97 | 82 | 96 | 98 | 85 | 77 | 98 | 87 | 90 | 82 | 92 |
| | N | I | G | E | | M | G | A | F | | N | G | | N | K | Y | T |
| | 26 | 6 | 12 | 9 | | 24 | 3 | 16 | 4 | | 11 | 20 | | 13 | 6 | 17 | 4 |
| | E | | S | | | | | | | | | | | | | | |
| | 9 | | 10 | | | | | | | | | | | | | | |
| | T | | | | | | | | | | | | | | | | |
| | 4 | | | | | | | | | | | | | | | | |
| 500,000 | | A | | Q | | | | | | | | | T | | E | | I |
| | | 3 | | 3 | | | | | | | | | 2 | | 2 | | 2 |
| 200,000,000 | M | | H | | G | | | V | | T | D | N | | | L | N | D |
| | 2 | | 1 | | 2 | | | 2 | | 2 | 2 | 1 | | | 1 | 1 | 1 |
| | R | | | | | | | | | | K | S | | | H | | H |
| | 2 | | | | | | | | | | 1 | 1 | | | 1 | | 1 |
| | | | | | | | | | | | T | I | | | | | |
| | | | | | | | | | | | 1 | 1 | | | | | |

Peptide libraries based on the V3 region of HIV gp120 from subtype D were designed. The libraries had the structure:
X¹—C—X²—R—P—X³—X⁴—X⁵—X⁶—R—X⁷—X⁸—X⁹—X¹⁰—I—G—X¹¹—G—X¹²—X¹³—X¹⁴—X¹⁵—X¹⁶—T—X¹⁷—E—X¹⁸—G—X¹⁹—I—X²⁰—X²¹—A—X²²—C—X²³

where X¹-X²³ are defined according to Table 21.

TABLE 21
HIV gp120 V3 region, subtype D

| Library size < | X¹ | X² | X³ | X⁴ | X⁵ | X⁶ | X⁷ | X⁸ | X⁹ | X¹⁰ | X¹¹ | X¹² |
|---|---|---|---|---|---|---|---|---|---|---|---|---|
| 50,000 | N | T | Y | N | N | T | Q | S | T | H | P | Q |
| | 78 | 83 | 74 | 87 | 89 | 72 | 71 | 37 | 74 | 61 | 64 | 70 |
| | T | I | N | | | | R | R | I | P | L | R |
| | 22 | 17 | 26 | | | | 29 | 35 | 26 | 19 | 16 | 30 |
| | | | | | | | | G | | | | |
| | | | | | | | | 28 | | | | |
| 500,000 | | | | K | | I | | | | | S | |
| | | | | 13 | | 16 | | | | | 11 | |
| | | | | | | K | | | | | | |
| | | | | | | 12 | | | | | | |
| 200,000,000 | | | | | K | | | | | S | Q | |
| | | | | | 11 | | | | | 10 | 8 | |
| | | | | | | | | | | R | | |
| | | | | | | | | | | 9 | | |

| Library size < | X¹³ | X¹⁴ | X¹⁵ | X¹⁶ | X¹⁷ | X¹⁸ | X¹⁹ | X²⁰ | X²¹ | X²² | X²³ |
|---|---|---|---|---|---|---|---|---|---|---|---|
| 50,000 | A | L | Y | T | I | I | D | R | Q | H | N |
| | 92 | 80 | 80 | 92 | 82 | 83 | 84 | 92 | 84 | 67 | 82 |
| | | Y | F | | K | | N | | K | Y | |
| | | 20 | 20 | | 18 | | 16 | | 16 | 33 | |
| 500,000 | | | | | | | | | | | |
| 200,000,000 | T | | | A | | T | | G | | | T |
| | 8 | | | 8 | | 10 | | 8 | | | 11 |
| | | | | | | K | | | | | K |
| | | | | | | 8 | | | | | 8 |

Peptide libraries based on the V3 region of HIV gp120 from subtype F were designed. The libraries had the structure:
X¹—C—T—R—P—X²—N—N—X³—R—K—X⁴—I—X⁵—L—P—G—P—X⁶—X⁷—X⁸—X⁹—X¹⁰—X¹¹—X¹²—X¹³—I—X¹⁴—G—X¹⁵—I—R—X¹⁶—A—X¹⁷—C—X¹⁸

where X¹-X¹⁸ are defined according to Table 22.

TABLE 22
HIV gp120 V3 region, subtype F

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ |
|---|---|---|---|---|---|---|---|---|---|---|---|---|---|---|---|---|---|---|
| 50,000 | N | N | T | S | H | Q | A | F | Y | A | T | G | D | I | D | K | H | N |
| | 93 | 96 | 97 | 91 | 73 | 69 | 87 | 97 | 92 | 73 | 95 | 92 | 87 | 96 | 91 | 92 | 86 | 83 |
| | T | | | G | R | R | V | | | T | A | D | A | T | N | Q | Y | I |
| | 7 | | | 6 | 10 | 28 | 10 | | | 27 | 5 | 5 | 8 | 4 | 9 | 8 | 14 | 11 |
| | | | | | Q | | | | | | | | N | | | | | |
| | | | | | 6 | | | | | | | | 5 | | | | | |
| 500,000 | | S | | | P | | | | H | | | | | | | | | |
| | | 4 | | | 4 | | | | 4 | | | | | | | | | |
| | | | | | Y | | | | F | | | | | | | | | |
| | | | | | 4 | | | | 4 | | | | | | | | | |
| 200,000,000 | | | I | R | S | H | T | I | | | | S | | | | | | D |
| | | | 3 | 3 | 3 | 3 | 3 | 3 | | | | 3 | | | | | | 3 |
| | | | | | | | | | | | | | | | | | | K |
| | | | | | | | | | | | | | | | | | | 3 |

Peptide libraries based on the V3 region of HIV gp120 from subtype G were designed. The libraries had the structure:
X¹—C—X²—R—P—X³—N—N—T—R—K—S—X⁴—X⁵—X⁶—G—X⁷—X⁸—X⁹—X¹⁰—X¹¹—X¹²—X¹³—T—X¹⁴—I—X¹⁵—G—X¹⁶—I—R—X¹⁷—A—X¹⁸—C—X¹⁹

where X¹-X¹⁹ are defined according to Table 23.

TABLE 23
HIV gp120 V3 region, subtype G

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ |
|---|---|---|---|---|---|---|---|---|---|---|---|---|---|---|---|---|---|---|---|
| 50,000 | N | T | N | I | T | F | P | G | Q | A | F | Y | A | G | I | D | Q | H | N |
| | 44 | 90 | 88 | 98 | 32 | 66 | 93 | 98 | 86 | 76 | 93 | 98 | 95 | 91 | 98 | 76 | 94 | 83 | 96 |
| | I | I | S | | R | I | | | R | T | I | | T | D | | N | | Y | |
| | 19 | 10 | 12 | | 25 | 34 | | | 7 | 17 | 5 | | 5 | 5 | | 24 | | 15 | |
| | T | | | | H | | | | | V | | | | | | | | | |
| | 15 | | | | 22 | | | | | 7 | | | | | | | | | |
| | M | | | | P | | | | | | | | | | | | | | |
| | 12 | | | | 15 | | | | | | | | | | | | | | |
| | R | | | | N | | | | | | | | | | | | | | |
| | 9 | | | | 5 | | | | | | | | | | | | | | |
| 500,000 | | | | | | | T | | H | | | | | S | | | | | K |
| | | | | | | | 5 | | 5 | | | | | 4 | | | | | 2 |
| 200,000,000 | | | | K | | | L | R | G | | S | H | | | T | | L | F | T |
| | | | | 2 | | | 2 | 2 | 2 | | 2 | 2 | | | 2 | | 2 | 2 | 2 |
| | | | | | | | | | | | | | | | | | K | | |
| | | | | | | | | | | | | | | | | | 2 | | |
| | | | | | | | | | | | | | | | | | P | | |
| | | | | | | | | | | | | | | | | | 2 | | |

Chimeric Peptide Libraries—V4 region of HIV gp120

Peptide libraries based on the V4 region of HIV gp120 from subtypes A, B, C, D, F and G were designed. The libraries had the structure:
C—N—T—T—X¹—L—F—N—S—T—W—X²—X³—X⁴—X⁵—W—X⁶—T—X⁷—X⁸—X⁹—X¹⁰—X¹¹—X¹²—X¹³—X¹⁴—X¹⁵—X¹⁶—X¹⁷—I—X¹⁸—L—X¹⁹—C

where X¹-X¹⁹ are defined according to Table 24.

TABLE 24
HIV gp120 V4 region, subtypes ABCDFG

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ |
|---|---|---|---|---|---|---|---|---|---|---|---|---|---|---|---|---|---|---|---|
| 50,000 | Q | N | N | S | T | N | E | G | S | N | N | T | E | G | N | D | T | T | P |
| | 67 | 52 | 78 | 61 | 90 | 37 | 58 | 70 | 79 | 86 | 59 | 90 | 67 | 78 | 70 | 63 | 65 | 85 | 84 |
| | K | F | | N | | S | | E | L | | S | | | S | S | N | N | I | Q |
| | 33 | 18 | | 22 | | 33 | | 18 | 21 | | 24 | | | 22 | 30 | 13 | 25 | 15 | 16 |
| | | T | | G | | | | | | | D | | | | | | | | |
| | | 16 | | 17 | | | | | | | 18 | | | | | | | | |
| | | V | | | | | T | S | | D | | | | | | | | | |
| | | 14 | | | | | 15 | 12 | | 14 | | | | | | | | | |
| 500,000 | | | | | | | N | | | | | | | | | | | | |
| | | | | | | | 14 | | | | | | | | | | | | |
| | | | | | | | K | | | | | | | | | | | | |
| | | | | | | | 13 | | | | | | | | | | | | |
| 200,000,000 | | | S | | A | D | | | | | | N | K | | | G | I | | |
| | | | 11 | | 10 | 11 | | | | | | 10 | 12 | | | 12 | 9 | | |
| | | | G | | | V | | | | | | | T | | | S | | | |
| | | | 10 | | | 9 | | | | | | | 11 | | | 12 | | | |
| | | | | | | G | | | | | | | N | | | | | | |
| | | | | | | 8 | | | | | | | 10 | | | | | | |

Peptide libraries based on the V4 region of HIV gp120 from subtype A were designed. The libraries had the structure:
C—X¹—T—S—X²—L—F—N—X³—T—W—X⁴—X⁵—X⁶—X⁷—X⁸—X⁹—X¹⁰—X¹¹—T—X¹²—X¹³—X¹⁴—X¹⁵—X¹⁶—I—X¹⁷—L—X¹⁸—C

where X¹-X¹⁸ are defined according to Table 25.

TABLE 25
HIV gp120 V4 region, subtype A

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ |
|---|---|---|---|---|---|---|---|---|---|---|---|---|---|---|---|---|---|---|
| 50,000 | N | G | S | T | N | I | Q | E | S | N | S | E | S | N | D | T | T | P |
| | 87 | 56 | 90 | 43 | 62 | 26 | 48 | 58 | 85 | 91 | 85 | 60 | 80 | 85 | 72 | 66 | 91 | 66 |
| | | D | | N | S | T | T | S | L | | | K | L | | G | N | | Q |
| | | 22 | | 32 | 20 | 25 | 41 | 17 | 15 | | | 17 | 20 | | 28 | 28 | | 34 |
| | | | | E | | M | | G | | | | G | | | | | | |
| | | | | 25 | | 23 | | 14 | | | | 16 | | | | | | |
| | | | | | | G | | | | | | | | | | | | |
| | | | | | | 14 | | | | | | | | | | | | |
| 500,000 | D | | G | | A | N | E | V | | | | | | | | | | |
| | 13 | | 10 | | 10 | 12 | 10 | 10 | | | | | | | | | | |
| 200,000,000 | | S | | | D | | | | | D | D | N | | S | | K | I | |
| | | 8 | | | 8 | | | | | 9 | 9 | 7 | | 9 | | 6 | 9 | |
| | | K | | | | | | | | | N | | | D | | | | |
| | | 7 | | | | | | | | | 8 | | | 6 | | | | |
| | | N | | | | | | | | | I | | | | | | | |
| | | 7 | | | | | | | | | 7 | | | | | | | |

Peptide libraries based on the V4 region of HIV gp120 from subtype B were designed. The libraries had the structure:
C—N—T—T—X¹——F—N—S—T—W—X²—X³—X⁴—X⁵—X⁶—X⁷—T—X⁸—X⁹—X¹⁰—X¹¹—X¹²—X¹³—X¹⁴—G—X¹⁵—X¹⁶—X¹⁷—I—X¹⁸—L—X¹⁹—C

where X¹-X¹⁹ are defined according to Table 26.

TABLE 26
HIV gp120 region V4, subtype B

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ | X¹⁸ | X¹⁹ |
|---|---|---|---|---|---|---|---|---|---|---|---|---|---|---|---|---|---|---|---|
| 50,000 | Q | N | N | S | T | W | N | E | G | S | N | N | T | E | N | D | T | T | P |
| | 64 | 55 | 89 | 62 | 90 | 90 | 43 | 61 | 74 | 79 | 85 | 59 | 89 | 69 | 72 | 65 | 63 | 83 | 86 |
| | K | T | | N | | | S | T | E | L | | D | | K | S | N | N | I | |
| | 27 | 16 | | 20 | | | 34 | 15 | 17 | 21 | | 16 | | 12 | 28 | 13 | 27 | 17 | |
| | | F | | G | | | | K | | | | S | | | | | | | |
| | | 16 | | 17 | | | | 15 | | | | 16 | | | | | | | |
| 500,000 | | V | | | | | D | | | | D | | | | | S | | | Q |
| | | 13 | | | | | 13 | | | | 15 | | | | | 11 | | | 14 |
| 200,000,000 | P | | G | | A | G | V | N | R | | | G | N | T | | G | I | | |
| | 9 | | 11 | | 10 | 10 | 10 | 9 | 10 | | | 9 | 11 | 10 | | 11 | 9 | | |
| | | | | | | | | | | | | | | N | | | | | |
| | | | | | | | | | | | | | | 9 | | | | | |

Peptide libraries based on the V4 region of HIV gp120 from subtype C were designed. The libraries had the structure:
C—N—T—S—X¹—L—F—N—X²—T—Y—X³—X⁴—X⁵—X⁶—X⁷—X⁸—X⁹—X¹⁰—X¹¹—X¹²—X¹³—X¹⁴—X¹⁵—X¹⁶—L—X¹⁷—C

where X¹-X¹⁷ are defined according to Table 27.

TABLE 27
HIV gp 120 V4 region, subtype C

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ | X¹⁷ |
|---|---|---|---|---|---|---|---|---|---|---|---|---|---|---|---|---|---|
| 50,000 | K | S | N | N | G | T | Y | N | S | T | E | S | N | S | T | T | P |
| | 36 | 65 | 47 | 58 | 63 | 91 | 50 | 78 | 58 | 79 | 45 | 59 | 78 | 70 | 71 | 92 | 77 |
| | G | G | M | S | S | | S | | G | N | S | D | S | | N | | Q |
| | 24 | 35 | 27 | 25 | 18 | | 20 | | 19 | 21 | 28 | 15 | 22 | | 16 | | 23 |
| | N | | S | P | | | | | | | N | | | | | | |
| | 20 | | 18 | 17 | | | | | | | 15 | | | | | | |
| | | | | | | | F | | | | | | | A | I | | |
| | | | | | | | 16 | | | | | | | 16 | 13 | | |
| 500,000 | | | | | | | N | | | | | | | E | | | |
| | | | | | | | 14 | | | | | | | 15 | | | |
| 200,000,000 | S | | R | | D | L | | I | N | | G | N | | | | I | |
| | 10 | | 8 | | 10 | 9 | | 11 | 13 | | 12 | 11 | | | | 8 | |
| | D | | | | N | | | T | D | | | V | | | | | |
| | 9 | | | | 8 | | | 11 | 10 | | | 9 | | | | | |
| | | | | | | | | | | | | G | | | | | |
| | | | | | | | | | | | | 7 | | | | | |

Peptide libraries based on the V4 region of HIV gp120 from subtype D were designed. The libraries had the structure:
C—X¹—T—S—X²—L—F—N—X³—X⁴—X⁵—X⁶—X⁷—X⁸—X⁹—X¹⁰—X¹¹—X¹²—X¹³—X¹⁴—I—X¹⁵—C

where X¹-X¹⁵ are defined according to Table 28

TABLE 28
HIV gp 120 V4 region, subtype D

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ |
|---|---|---|---|---|---|---|---|---|---|---|---|---|---|---|---|
| 50,000 | N | K | S | T | W | D | N | N | T | G | D | N | I | T | P |
| | 94 | 62 | 83 | 92 | 85 | 40 | 47 | 32 | 78 | 43 | 60 | 74 | 70 | 54 | 67 |
| | | G | | | | N | T | G | | W | N | T | M | N | S |
| | | 26 | | | | 26 | 42 | 26 | | 25 | 17 | 26 | 30 | 24 | 21 |
| | | N | | | | S | | S | | N | S | | | I | Q |
| | | 13 | | | | 23 | | 23 | | 17 | 12 | | | 13 | 11 |
| | | | | | | | | E | | T | | | | | |
| | | | | | | | | 12 | | 10 | | | | | |
| 500,000 | | | | | L | K | | | N | | G | | | K | |
| | | | | | 11 | 9 | | | 8 | | 7 | | | 9 | |
| | | | | | | | | | L | | | | | | |
| | | | | | | | | | 8 | | | | | | |
| 200,000,000 | D | | R | E | Y | G | I | K | E | D | V | | | | |
| | 6 | | 7 | 5 | 4 | 4 | 6 | 7 | 5 | 5 | 4 | | | | |
| | | | N | V | | I | D | | | | | | | | |
| | | | 6 | 3 | | 3 | 2 | | | | | | | | |
| | | | G | | | | K | | | | | | | | |
| | | | 5 | | | | 2 | | | | | | | | |

Peptide libraries based on the V4 region of HIV gp120 from subtype F were designed. The libraries had the structure:
X¹—X²—T—X³—X⁴—L—F—X⁵—X⁶—X⁷—X⁸—X⁹—X¹⁰—X¹¹—X¹²—X¹³—I—X¹⁴—L—X¹⁵—C

where X¹-X¹⁵ are defined according to Table 29.

TABLE 29
HIV gp120 V4 region, subtype F

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ |
|---|---|---|---|---|---|---|---|---|---|---|---|---|---|---|---|
| 50,000 | C | N | S | G | N | D | T | N | D | T | N | G | T | T | P |
| | 95 | 91 | 91 | 53 | 85 | 44 | 92 | 81 | 46 | 84 | 86 | 53 | 81 | 82 | 89 |
| | | D | | E | T | N | | T | N | A | T | D | N | I | L |
| | | 9 | | 20 | 9 | 31 | | 6 | 28 | 10 | 6 | 32 | 8 | 18 | 8 |
| | | | | K | | S | | | S | | | S | I | | |
| | | | | 10 | | 17 | | | 10 | | | 9 | 6 | | |
| | | | | Q | | | | | | | | | | | |
| | | | | 8 | | | | | | | | | | | |
| 500,000 | | | D | I | K | A | | | G | | | N | P | | |
| | | | 6 | 6 | 6 | 5 | | | 5 | | | 6 | 5 | | |
| 200,000,000 | Y | | A | S | | H | A | E | M | N | K | | | | Q |
| | 5 | | 3 | 3 | | 3 | 4 | 5 | 4 | 3 | 4 | | | | 3 |
| | | | | | | | V | I | K | I | D | | | | |
| | | | | | | | 4 | 5 | 3 | 3 | 4 | | | | |
| | | | | | | | | A | I | | | | | | |
| | | | | | | | | 3 | 3 | | | | | | |

Peptide libraries based on the V4 region of HIV gp120 from subtype G were designed. The libraries had the structure:
C—N—T—S—X¹—L—F—X²—X³—X⁴—X⁵—X⁶—X⁷—X⁸—X⁹—X¹⁰—X¹¹—X¹²—X¹³—X¹⁴—I—X¹⁵—L—X¹⁶—C

where X¹-X¹⁶ are defined according to Table 30.

TABLE 30
HIV gp120 V4 region, subtype G

| Library size < | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ | X¹⁶ |
|---|---|---|---|---|---|---|---|---|---|---|---|---|---|---|---|---|
| 50,000 | G | N | N | S | N | S | N | T | S | N | N | N | T | T | T | P |
| | 54 | 86 | 54 | 88 | 63 | 57 | 61 | 39 | 88 | 35 | 61 | 74 | 45 | 78 | 87 | 95 |
| | K | | S | | S | D | S | I | | E | S | D | D | N | | |
| | 26 | | 25 | | 16 | 22 | 34 | 23 | | 29 | 27 | 15 | 34 | 15 | | |
| | | | E | | | N | | N | | K | E | | G | | | |
| | | | 21 | | | 20 | | 14 | | 14 | 12 | | 21 | | | |
| | | | | | | | | E | | T | | | | | | |
| | | | | | | | | 12 | | 14 | | | | | | |
| 500,000 | E | | | | K | | | | | A | | S | | | | |
| | 11 | | | | 12 | | | | | 8 | | 11 | | | | |
| | Q | | | | D | | | | | | | | | | | |
| | 10 | | | | 9 | | | | | | | | | | | |
| 200,000,000 | | R | | N | | | D | A | A | | | | | I | I | Q |
| | | 8 | | 6 | | | 5 | 7 | 7 | | | | | 6 | 7 | 5 |
| | | T | | E | | | | R | N | | | | | | M | |
| | | 6 | | 6 | | | | 4 | 6 | | | | | | 6 | |

Chimeric Peptide Libraries—V5 region of HIV gp120

Peptide libraries based on the V5 region of HIV gp120 from subtypes A, B, C, D, F, and G were designed. The libraries had the structure:
X¹—X²—X³—X⁴—X⁵—X⁶—E—X⁷—F—R—P—X⁸

where X¹-X⁸ are defined according to Table 31.

TABLE 31
HIV gp120 V5 region, subtypes ABCDFG

| Library size < | X¹ | X² | X³ | X⁴ | X⁵ | X⁶ | X⁷ | X⁸ |
|---|---|---|---|---|---|---|---|---|
| 50,000 | N | S | N | N | E | T | I | G |
| | 75 | 42 | 42 | 68 | 35 | 75 | 53 | 99 |
| | D | N | T | S | T | N | T | |
| | 9 | 25 | 36 | 18 | 23 | 20 | 47 | |
| | G | E | D | E | N | P | | |
| | 6 | 16 | 13 | 6 | 18 | 2 | | |
| | T | G | K | G | G | I | | |
| | 5 | 10 | 5 | 5 | 13 | 2 | | |
| | K | K | G | K | K | | | |
| | 3 | 5 | 4 | 3 | 10 | | | |
| | P | I | | | | | | |
| | 2 | 1 | | | | | | |
| 500,000 | | | | | M | E | | E |
| | | | | | 1 | 1 | | 1 |
| | | | | | H | | | |
| | | | | | 1 | | | |

Peptide libraries based on the V5 region of HIV gp120 from subtype A were designed. The libraries had the structure:
X¹—X²—X³—X⁴—X⁵—X⁶—X⁷—X⁸—X⁹—R—P—X¹⁰

where X¹-X¹⁰ are defined according to Table 32.

TABLE 32
HIV gp120 V5 region, subtype A

| Library size < | X¹ | X² | X³ | X⁴ | X⁵ | X⁶ | X⁷ | X⁸ | X⁹ | X¹⁰ |
|---|---|---|---|---|---|---|---|---|---|---|
| 50,000 | G | N | N | S | T | N | E | T | F | G |
| | 31 | 60 | 76 | 76 | 57 | 61 | 99 | 85 | 99 | 78 |
| | N | T | S | N | E | T | | I | | I |
| | 24 | 22 | 17 | 16 | 16 | 27 | | 15 | | 16 |
| | D | G | D | R | N | E | | | | T |
| | 24 | 6 | 3 | 4 | 10 | 7 | | | | 4 |
| | S | D | | | A | P | | | | |
| | 8 | 5 | | | 9 | 3 | | | | |
| | K | I | | | I | | | | | |
| | 6 | 4 | | | 4 | | | | | |
| | V | K | | | | | | | | |
| | 6 | 3 | | | | | | | | |
| 500,000 | | | | G | K | K | Q | | S | R |
| | | | | 2 | 2 | 2 | 1 | | 1 | 2 |
| | | | | | M | | | | | |
| | | | | | 1 | | | | | |
| 200,000,000 | | | R | E | P | | | | | |
| | | | 1 | 1 | 1 | | | | | |
| | | | G | F | | | | | | |
| | | | 1 | 1 | | | | | | |
| | | | I | | | | | | | |
| | | | 1 | | | | | | | |
| | | | H | | | | | | | |
| | | | 1 | | | | | | | |

Peptide libraries based on the V5 region of HIV gp120 from subtype B were designed. The libraries had the structure:
X¹—X²—X³—X⁴—X⁵—X⁶—E—X⁷—F—R—P—G

where X¹-X⁷ are defined according to Table 33.

TABLE 33
HIV gp120 V5 region, subtype B

| Library size < | X¹ | X² | X³ | X⁴ | X⁵ | X⁶ | X⁷ |
|---|---|---|---|---|---|---|---|
| 50,000 | N | S | N | N | E | T | I |
| | 78 | 44 | 40 | 69 | 37 | 77 | 57 |
| | D | N | T | S | T | N | T |
| | 9 | 25 | 37 | 16 | 21 | 19 | 43 |
| | G | E | D | G | N | P | |
| | 4 | 16 | 15 | 6 | 18 | 2 | |
| | T | G | K | E | G | I | |
| | 4 | 9 | 5 | 6 | 13 | 2 | |
| | K | K | G | K | K | | |
| | 3 | 6 | 4 | 3 | 10 | | |
| | P | M | | | M | | |
| | 2 | 1 | | | 2 | | |

Peptide libraries based on the V5 region of HIV gp120 from subtype C were designed. The libraries had the structure:
X¹—X²—X³—X⁴—X⁵—E—X⁶—X⁷—X⁸—X⁹—X¹⁰

where X¹-X¹⁰ are defined according to Table 34.

TABLE 34
HIV gp120 V5 region, subtype C

| Library size < | X¹ | X² | X³ | X⁴ | X⁵ | X⁶ | X⁷ | X⁸ | X⁹ | X¹⁰ |
|---|---|---|---|---|---|---|---|---|---|---|
| 50,000 | E | T | N | D | T | T | F | R | P | G |
| | 45 | 44 | 81 | 28 | 85 | 55 | 99 | 98 | 99 | 89 |
| | N | N | T | N | N | I | | | | E |
| | 18 | 16 | 13 | 26 | 6 | 42 | | | | 5 |
| | T | D | D | T | E | K | | | | I |
| | 12 | 13 | 4 | 85 | 5 | 2 | | | | 2 |
| | G | G | | K | G | | | | | Q |
| | 11 | 13 | | 6 | 2 | | | | | 2 |
| | K | K | | G | I | | | | | T |
| | 7 | 7 | | 9 | 2 | | | | | 2 |
| | L | I | | | | | | | | |
| | 5 | 5 | | | | | | | | |
| 500,000 | P | P | G | I | | | | Q | T | |
| | 2 | 2 | 2 | 2 | | | | 1 | 1 | |
| 200,000,000 | | | | | | N | I | S | | |
| | | | | | | 1 | 1 | 1 | | |

Peptide libraries based on the V5 region of HIV gp120 from subtype D were designed. The libraries had the structure:
X¹—X²—X³—X⁴—X⁵—X⁶—X⁷—E—X⁸—X⁹—R—X¹⁰-X¹¹

where X¹-X¹¹ are defined according to Table 35.

TABLE 35
HIV gp120 V5 region, subtype D

| Library size < | X¹ | X² | X³ | X⁴ | X⁵ | X⁶ | X⁷ | X⁸ | X⁹ | X¹⁰ | X¹¹ |
|---|---|---|---|---|---|---|---|---|---|---|---|
| 50,000 | N | N | N | S | N | Q | N | T | F | P | G |
| | 58 | 92 | 44 | 70 | 52 | 50 | 78 | 94 | 97 | 98 | 99 |
| | T | G | S | N | S | E | S | I | I | | |
| | 13 | 3 | 40 | 25 | 32 | 22 | 18 | 6 | 3 | | |
| | A | D | D | | R | H | | | | | |
| | 13 | 3 | 6 | | 7 | 12 | | | | | |
| | K | | G | | G | S | | | | | |
| | 9 | | 6 | | 4 | 10 | | | | | |
| | E | | | | H | P | | | | | |
| | 3 | | | | 3 | 3 | | | | | |
| | H | | | | | | | | | | |
| | 3 | | | | | | | | | | |
| 500,000 | | | I | G | D | R | H | | | T | |
| | | | 2 | 2 | 2 | 2 | 2 | | | 2 | |
| | | | H | R | | | R | | | | |
| | | | 2 | 2 | | | 2 | | | | |
| 200,000,000 | Y | H | | D | | G | | | | | Q |
| | 1 | 1 | | 1 | | 1 | | | | | 1 |
| | | K | | | | | | | | | |
| | | 1 | | | | | | | | | |

Peptide libraries based on the V5 region of HIV gp120 from subtype F were designed. The libraries had the structure:
X¹—X²—X³—X⁴—X⁵—X⁶—X⁷—F—R—P—X⁸

where X¹-X⁸ are defined according to Table 36.

TABLE 36
HIV gp120 V5 region, subtype F

| Library size < | X¹ | X² | X³ | X⁴ | X⁵ | X⁶ | X⁷ | X⁸ |
|---|---|---|---|---|---|---|---|---|
| 50,000 | N | N | N | T | N | E | T | G |
| | 43 | 65 | 46 | 51 | 60 | 97 | 83 | 80 |
| | L | D | S | K | S | Q | I | E |
| | 30 | 14 | 35 | 14 | 29 | 3 | 17 | 10 |
| | E | G | G | D | D | | | I |
| | 16 | 7 | 7 | 14 | 7 | | | 7 |
| | S | K | E | N | I | | | Q |
| | 8 | 7 | 6 | 14 | 4 | | | 3 |
| | K | T | K | G | | | | |
| | 4 | 7 | 6 | 7 | | | | |

Peptide libraries based on the V5 region of HIV gp120 from subtype G were designed. The libraries had the structure:
X¹—X²—X³—X⁴—X⁵—X⁶—X⁷—X⁸—X⁹—X¹⁰—X¹¹—X¹²

where X¹-X¹² are defined according to Table 37.

TABLE 37
HIV gp120 V5 region, subtype G

| Library size < | X¹ | X² | X³ | X⁴ | X⁵ | X⁶ | X⁷ | X⁸ | X⁹ | X¹⁰ | X¹¹ | X¹² |
|---|---|---|---|---|---|---|---|---|---|---|---|---|
| 50,000 | N | N | N | S | T | N | E | T | F | R | P | G |
| | 40 | 78 | 75 | 53 | 85 | 63 | 98 | 82 | 91 | 93 | 87 | 88 |
| | A | T | T | N | G | T | | I | I | | A | R |
| | 18 | 9 | 10 | 35 | 7 | 25 | | 17 | 5 | | 7 | 8 |
| | T | A | D | E | | E | | | | | T | |
| | 16 | 8 | 10 | 4 | | 6 | | | | | 4 | |
| | D | | | A | | | | | | | | |
| | 15 | | | 4 | | | | | | | | |
| | R | | | | | | | | | | | |
| | 9 | | | | | | | | | | | |
| 500,000 | | | A | | E | G | | | N | E | | |
| | | | 3 | | 3 | 4 | | | 2 | 4 | | |
| | | | | | | | | | T | S | | |
| | | | | | | | | | 2 | 2 | | |
| 200,000,000 | Y | K | K | I | K | K | N | D | | G | D | E |
| | 2 | 2 | 2 | 2 | 2 | 1 | 1 | 1 | | 1 | 1 | 1 |
| | | D | | K | N | F | S | | | | K | W |
| | | 2 | | 1 | 2 | 1 | 1 | | | | 1 | 1 |
| | | P | | H | F | | | | | | | Y |
| | | 1 | | 1 | 1 | | | | | | | 1 |
| | | | | | | | | | | | | V |
| | | | | | | | | | | | | 1 |

Synthesis and Antigenic Properties of a VCPL

A peptide library mimicking the naturally occurring diversity of sequences in the main hypervariable V3 region of the HIV-1 (subtype B) gp120 envelope protein was designed. The identity and frequency of the amino acids in the library were chosen based on sequence alignments and antigenic similarity. HIV-1 sequences are available from the HIV Sequence Database (hiv-web.lanl.gov). The peptide library had the structure:
N—N—N—T—R—K—X¹—I—X²—X³—X⁴—X⁵—G—X⁶—X⁷—X⁸—Y—X⁹—T—G—X¹⁰I—I—G—X¹¹—R—Q

where X¹- X¹¹ were defined according to Table 38.

TABLE 38

| X¹ | X² | X³ | X⁴ | X⁵ | X⁶ | X⁷ | X⁸ | X⁹ | X¹⁰ | X¹¹ |
|---|---|---|---|---|---|---|---|---|---|---|
| S | H | I | G | P | R | A | F | T | E | D |
| 75 | 60 | 82 | 97 | 95 | 77 v | 93 | 76 | 58 | 39 | 88 |
| G | P | M | A | W | Q | T | W | A | D | N |
| 18 | 18 | 12 | 3 | 5 | 9 | 7 | 15 | 42 | 28 | 12 |
| R | N | | | | K | | L | | Q | |
| 7 | 11 | | | | 8 | | 9 | | 24 | |
| | S | | | | S | | | | R | |
| | 11 | | | | 3 | | | | 9 | |
| | | | | | G | | | | | |
| | | | | | 3 | | | | | |

The peptides of the library were synthesized on Applied Biosystems 430A and Vega Coupler C250 automatic synthesizers with the use of the Boc/Bzl strategy of peptide chain elongation. A styrene copolymer with 1% divinylbenzene was used as the polymer support, and a phenacylamidomethyl (PAM) group served as the anchor group (Sparrow, J. T. J. Org. Chem. 1976, 41:1350-1353, which is incorporated by reference in its entirety). Condensation was done with 1-hydroxybenzotriazole esters prepared directly before use by mixing a 3-fold molar excess relative to the polymers of the Boc product of amino acid, 1-hydroxybenzotriazole and diisopropylcarbodiimide. The completeness of the condensation was monitored (Kaiser, E. 398, which is incorporated by reference in its entirety). In the event of a positive ninhydrin test after neutralization of the peptidyl polymer, condensation was repeated. If the test was negative, then the temporary Boc protective groups were removed with undiluted trifluoroacetic acid, and neutralization was performed by adding N,N′-diisopropylethylamine directly to the reaction mixture (Schnolzer, M. et al. Int. J. Pept. Prot Res. 1992, 40: 180-183, which is incorporated by reference in its entirety). After removal of the Boc groups the following amino acid residue was attached. Permanent protective groups used for side chains were: 2-chlorobenzyloxycarbonyl groups for lysine, dichlorobenzyl for tyrosine, the corresponding benzyl esters for aspartic acid, glutamic acid, serine and threonine, formyl for tryptophan, tosyl for arginine, and methionine was introduced in the form of the corresponding sulfoxide. Upon completion of assembly of the protective polypeptide chain, the end products were deblocked with a one-time cleavage from the polymer with the aid of anhydrous hydrogen fluoride in the presence of scavengers (Tam, J. P. et al. J. Am. Chem. Soc. 1983, 105: 6442-6444, which is incorporated by reference in its entirety).

During the synthesis of peptide libraries a mixture of protected amino acid residues was added in the condensation stage in variable positions relative to the peptidyl polymer in a certain quantitative relationship, which provided a given incorporation of different amino acid residues to the growing polypeptide chain. Because different amino acids have different rates of amino group acylation, a number of model compounds were synthesized and reaction rate coefficients were derived. The Boc products of the amino acids which were introduced into the mixture, were used in ratios adjusted for the reaction rate coefficients, to provide peptide products having the desired ratios (as shown in Table 38) in the variable positions.

The peptide library was purified by gel permeation chromatography using Sepharose G-50 and 50% acetic acid as the eluent. The synthesized compounds were characterized by analytical single-photon high-performance liquid chromatography (Gilson chromatograph, France; Xterra RP18 column, 125 Å, 3.9×150 mm, 5 μm, flow rate 1 mL/min, eluent 0.1% trifluoroacetic acid, 10-40% and 20-40% acetonitrile gradient in 16 min for pentarphin and cyclopentarphin, respectively), and by amino acid analysis (hydrolysis 6N HCl, 24 hours, 110° C.; LKB amino acid analyzer, Alpha Plus, Sweden). The purity of the end products according to analytic HPLC was not less than 95%. The amino acid composition of peptide hydrolysates (6N HCl, 120° C., 24 hours, LKB 4151 analyzer, Alpha Plus) corresponded to the theoretical.

BALB/c line mice ages 5-6 weeks were immunized 3 times intraperitoneally with the peptide library diluted in a phosphate buffer solution with Complete Freund's Adjuvant (CFA). The mice were administered 0.2 mL peptide solution in a dose of 50 μg/head. The second and third immunization were conducted 2 and 4 weeks after the first immunization, respectively. A control group of animals were administered a peptide solution mimicking the antigenic portion of protein E of the tick encephalitis virus (peptide sequence: KRDQSDRGWGNHAGLFGKGSIVT) according to the same scheme and in the same doses. Ten days after the last immunization a blood sample was taken from the retroorbital vein of the animals for determination of the titer of specific antibodies in the serum.

The following antigens were immobilized in separate wells of microtiter plates for immunoenzyme analysis (Medpolimer, Moscow): the chimeric-peptide library, and each of 15 synthetic peptides representing the amino acid sequences of the V3 region of gp120 from different HIV-1 subtypes. The 15 synthetic peptides represent the consensus sequences of appropriate subtypes. Where several peptides are present from one subtype, they represent different variants.

The antigens were dissolved in 0.05 M carbonate-bicarbonate buffer solution, pH 9.6 to a final concentration of 10 μg/mL, placed in each well of the microtiter plate in 0.1 mL amounts and incubated overnight at a temperature of 37° C. The wells were washed 3 times with phosphate buffer solution with Tween 20, and non-specific binding was blocked with a 0.5% solution of bovine serum albumin, fraction 5 (Sigma). Mouse blood sera samples were serially diluted two-fold in a Tris-HCl buffer solution, and placed in 0.1 mL amounts into the wells with immobilized antigen and incubated at 37° C. for 30 min. After incubation the wells were washed 5 times with a phosphate buffer solution. The antigen-antibody complexes were determined by adding 0.1 mL quantities of horseradish peroxidase-conjugated goat antibodies against mouse IgG (Sigma) in phosphate buffer solution. Following incubation at 37° C. for 30 min, a substrate mixture containing orthophenylene diamine was added. The enzyme reaction was halted by the addition of a sulfuric acid solution, and the optical density at 492 nm was measured using a Multiscan EX microplate photometer. The results of titer determination of the antigen-specific antibodies in the mouse blood sera are presented in Table 39.

Immunization of mice with the chimeric-peptide library caused formation of serum antibodies specific both for the chimeric-peptide library itself (the immunizing antigens) and for peptides forming antigenic determinants in the V3 region of some known HIV-1 subtypes. In particular, the antibody titers for subtype B peptides were higher than for other subtypes, as would be expected for a peptide library design to mimic the diversity of subtype B and not other subtypes. The chimeric peptide library was immunogenic, and induced formation of antibodies that interact with antigenic determinants of a broad spectrum of HIV-1 variants.

TABLE 39

| Immobilized antigen | Mean titer of |
|---|---|
| (peptides corresponding to | specific |
| region V3 gp120 HIV-1, subtype) | antibodies |
| Chimeric-peptide library | 1:1600 |
| Peptide No 1, subtype A | 1:400 |
| Peptide No 2, subtype B | 1:3200 |
| Peptide No 3, subtype B | 1:1600 |
| Peptide No 4, subtype B | 1:800 |
| Peptide No 5, subtype B | 1:1600 |
| Peptide No 6, subtype B | 1:1600 |
| Peptide No 7, subtype C | 1:400 |
| Peptide No 8, subtype D | 1:400 |
| Peptide No 9, subtype E | 1:400 |
| Peptide No 10, subtype E | 1:200 |
| Peptide No 11, subtype F | 1:100 |
| Peptide No 12, subtype G | <1:100 |
| Peptide No 13, subtype H | <1:100 |
| Peptide No 14, subtype I | 1:100 |
| Peptide No 15, subtype J | 1:100 |
| Peptide of protein E of | <1:100 |
| the tick encephalitis virus | |
| (control) | |

The peptide library described above was tested as a diagnostic reagent in solid-phase immunoenzyme analysis (i.e., ELISA) of serum samples from 50 HIV-infected patients and 30 healthy donors for the purpose of determining specific antibodies. Preliminarily sera of HIV-infected patients were analyzed with the aid of a number of commercial test systems. In addition, the peptide library was assessed for its ability to detect antibodies to HIV-1 with the aid of standard sera panels anti-HIV-1 series 010 and series 007.

The chimeric-peptide library was dissolved in 0.05 M carbonate-bicarbonate buffer solution, pH 9.6 to a final concentration of 1.5 micrograms/mL. The solutions were added in 0.1 mL quantities to the wells of the microtiter plates and incubated at room temperature overnight, to immobilize the peptides in the wells. Then the wells were washed 3-5 times with a solution of FSB-T (phosphate-salt buffer solution containing Tween 20). Henceforth the method completely corresponds to the instructions for the use of the immunoenzyme test system for detecting antibodies to HIV-1 produced by DGUEPP “Vektor-BiAlgam” (Kol'tsovo, Novosibirsk Oblast).

Blood sera samples were diluted 20 fold in an FSB-T solution containing 0.2% casein and were placed in wells of the microtiter plate in 0.1 mL amounts and incubated at 37° C. for 30 min, to bind antibodies to the immobilized peptides. Then the wells were washed 5-7 times with FSB-T solution. To the wells were added 0.1 mL amounts of a monoclonal antibody to IgG conjugated to horseradish peroxidase in a dilution of 1:20 in FSB-T containing 0.2% casein. The microtiter plate was incubated at 37° C. for 30 min, then washed 5-7 times with an FSB-T solution. Next, 0.1 mL amounts of a solution of substrate mixture of 0.05% TMB and 0.005% hydrogen peroxide in 0.05 M phosphate-citrate buffer solution, pH 5.0 was added to the wells. The microtiter plate was kept at room temperature (18-22° C.) for 15-20 min in the dark. The reaction was stopped by addition of 0.05 mL amounts of 2 M sulfuric acid solution to the wells. Calculation of the results is accomplished by measurement of the optical density of the samples on a Multiscan-type automatic spectrophotometer at a wavelength of 450 nm.

As a control for ruling out non-specific results in setting up the reaction a substrate mixture (BCP+substrate) is used, along with a control conjugate (BCP+conjugate+substrate), and a control serum which does not contain antibodies to HIV according to data from testing in Russian and foreign third generation screening test systems.

The test sera are judged to have antibodies to HIV-1 if the values of the optical density (OD) of the corresponding investigative solution in the wells with the investigative sera exceed the critical level ODcrit, calculated according to the formula:
ODcrit=OD(C′)ₘₑₐₙ+0.2
where OD(C′)ₘₑₐₙ is the mean optical density for negative control sera.

The results of the study of the anti-HIV-1 standard panel sera series 010 and 007 with the use of the chimeric-peptide library showed an 81% sensitivity and 93% specificity. In addition, a study was undertaken of the effectiveness of the chimeric-peptide library for detection of specific antibodies to HIV-1 in blood sera samples with the use of 50 sera from HIV-infected patients and 30 sera from healthy donors. The results of the tests showed more than 80% detection of positive samples containing specific antibodies to HIV-1 antigens on the basis of results with commercial third generation test systems.

To test the immunogenicity of the chimeric peptide library in different antigen forms, the library was synthesized as linear peptides, a multiple antigen peptide of 4 linear peptides (MAP4), or multiple antigen peptide of 8 linear peptides (MAP8). Rabbits were immunized subcutaneously with 300 micrograms of the constructions described above mixed with either complete Freund's adjuvant (first immunization) or incomplete Freund's adjuvant (second and third immunizations) at 12 day intervals. At day 12 after the third immunization, the rabbits were bled and all blood sera were tested by ELISA against the linear, MAP4 and MAP8 antigens. Table 40 illustrates that immunization of rabbits with the HIV-1 V3 library in different antigen forms induces a strong humoral immune response to the highly variable V3 loop sequences from chimeric peptide library.

TABLE 40

| | Antibody titers in ELISA for different antigen forms | | |
|---|---|---|---|
| Immunizing dose | linear | MAP4 | MAP8 |
| linear | 5,120 | — | — |
| MAP4 | 81,920 | 163,840 | 163,840 |
| MAP8 | 10,240 | 81,920 | 81,920 |

The immunogenicity of the MAP4 chimeric peptide library in different formulations was tested. The library was formulated in Freund's adjuvant, artificial virus like particles (VLP) or in phosphate buffered solution (PBS). In addition, the MAP4 construction was modified with palmitic acid (MAP4-P) and formulated in liposomes or microemulsion. The liposomes included 100 micrograms of MAP4-P and 250 micrograms of dsRNA, and the microemulsion included 100 micrograms of MAP4-P and 100 micrograms of dsRNA.

The VLPs contain a DNA molecule covered with polypeptides carrying the MAP4 peptide library. The target polypeptides are exposed on the surface of the particle and are attached to DNA via spermidine-polygluquine conjugates. The dimensions of the particle will allow the target antigens to conjugate on the surface in copy numbers ranging from one to several hundred. The core of the VLP can contain DNA segments as large as 10,000 bp. See, for example, Lebedev, L. R. et al. (2000) Molecular genetics, microbiology and virology (Russian)(3), 36-40; Lebedev, L. R., et al. (2000). Molecular Biology (Russian) 34(3), 480-485; and Lebedev, L. R. et al. (2001) Biotechnology (Russian)(1), 3-12; and Sizov, A. A. et al. (2001) Biotechnology (Russian)(1), 13-18, each of which is incorporated by reference in its entirety.

The liposomes forms were prepared with the MAP4-P library. The palmitic acid modification increases the lipophilicity of the peptides. The liposomal compositions of the peptide were made by solubilization-injection. The size of the bulk of liposomes was within 200 nm.

The microemulsions were also made with the MAP4-P peptide construct and contained (in 1 mL) 100 micrograms of peptide, 250 micrograms of ridostin, and 800 micrograms of the commercial (ICN) cationic amphiphile dimethyldioctadecyl ammonium bromide (DDAB).

Mice (five mice in each group) were immunized intraperitoneally with the constructions described above three times at 12 days intervals. The data in Table 41 indicate the high diversity of individual immune response in conventional mice, but nevertheless strong humoral immune response was detected for all systems after 3^{rd} immunization, except for the peptide library solution in PBS. The antigen formulation of MAP4-P in liposomes induced high antibody levels after the second immunization.

TABLE 41

| | | | | AB titers in mouse serum in ELISA for | | | | | | | |
|---|---|---|---|---|---|---|---|---|---|---|---|
| | Antigen | Total | | MAP4 in solid phase | | | | | | AB Titer, | AB |
| Antigen | Delivery | Dose | Im. | Mouse # | | | | | AB titer, | Stand. | Titer, |
| Form | System | μg | No. | 1 | 2 | 3 | 4 | 5 | Average | Deviation | I95 |
| MAP4 | PBS | 40 | 2^{nd} | 0 | 0 | 0 | 0 | — | 0 | 0 | |
| | | 60 | 3^{rd} | 0 | 0 | 0 | 0 | — | 0 | 0 | |
| MAP4 | Freund's | 40 | 2^{nd} | 0 | 3200 | 1600 | 1600 | 12800 | 3840 | 5135 | 4501 |
| | adjuvant | 60 | 3^{rd} | 51200 | 12800 | 102400 | 25600 | 204800 | 79360 | 78070 | 68430 |
| MAP4-P | Liposome | 40 | 2^{nd} | 0 | 51200 | 51200 | 25600 | — | 32000 | 24510 | 24019 |
| | | 60 | 3^{rd} | 51200 | 102400 | 51200 | 102400 | — | 76800 | 29560 | 28969 |
| MAP4-P | Microemulsion | 40 | 2^{nd} | 0 | 0 | 6400 | 25600 | — | 8000 | 12115 | 11872 |
| | | 60 | 3^{rd} | 0 | 0 | 204800 | 51200 | — | 64000 | 96920 | 94980 |
| MAP4-P | VLP | 100 | 2^{nd} | 12800 | 12800 | 6400 | — | — | 10667 | 3695 | 4181 |
| | | 150 | 3^{rd} | 51200 | 204800 | 25600 | — | — | 93867 | 96920 | 109673 |

To determine the polarization of the immune response of mice immunized with the MAP4 library in different delivery systems described above, on day 10 after the 3^{rd} immunization, splenocytes were distinguished. The splenocytes were stimulated in vitro with the library of linear peptides. A number of splenocytes produced IFN-gamma or IL-4 cytokines, markers of Th1 or Th2 immune response, respectively, as measured by ELISPOT. The number of cells that produced one of two cytokines was noted, after stimulation or without stimulation (Table 42). The results presented in Table 42 show that immunization of mice with a solution of MAP4 peptide library with Freund's Adjuvant does not result in a major difference (significance level 0.05) in the number of stimulated cells from the control measurement (without stimulation), which confirms the absence of induction of the T-helper response with immunization by antigens in a mixture with Freund's Adjuvant. The highest number of cells producing IFN-gamma after stimulation in vitro using the linear peptide library was noted in mice immunized with a microemulsion containing the MAP4 library. The immunization using VLP resulted in the formation of a less pronounced immune response of the Th1 type. Comparison of the number of cells producing IL4 and IFN-gamma reveals that immunization of mice with the MAP4 library in VLPs results in a balanced T-helper response, i.e. the number of cells producing IFN-gamma and IL-4 does not significantly differ. On the other hand, immunization of mice with the same antigens in a microemulsion results in a significant predominance of the Th1 type immune response after stimulation with the library of linear peptides. Thus, the above example shows the possibility of inducing a strong T-helper response using the MAP4 library of chimeric peptides.

TABLE 42

| Antigen | Splenocytes | | | | | | | Average | | I95 of |
|---|---|---|---|---|---|---|---|---|---|---|
| delivery | stimulated in | Cell sample ## | | | | | | No. of | Standard | cell |
| system | vitro? | 1 | 2 | 3 | 4 | 5 | 6 | cells | Deviation | number |
| | | Number of cells producing IFN-gamma | | | | | | | | |
| | | Determined for 500,000 splenocytes in ELISPOT | | | | | | | | |
| VLP | Yes | 59 | 99 | 272 | — | 55 | 29 | 102.80 | 97.84 | 85.76 |
| | No | 2 | 5 | 1 | — | 3 | 5 | 3.20 | 1.79 | 1.57 |
| CFA/IFA | Yes | 6 | 3 | 33 | 37 | 12 | 10 | 16.83 | 14.47 | 11.58 |
| | No | 1 | 0 | 7 | 6 | 1 | 1 | 2.67 | 3.01 | 2.41 |
| Micro | Yes | 165 | 145 | 525 | 450 | 600 | 600 | 414.17 | 208.41 | 166.76 |
| emulsion | No | 4 | 0 | 1 | 2 | 3 | 1 | 1.83 | 1.47 | 1.18 |
| | | Number of cells producing IL-4. | | | | | | | | |
| | | Determined for 500,000 splenocytes in ELISPOT | | | | | | | | |
| VLP | Yes | 21 | 36 | 420 | 490 | 61 | 43 | 178.50 | 215.70 | 172.59 |
| | No | 11 | 8 | 9 | 13 | 3 | 0 | 7.33 | 4.93 | 3.94 |
| CFA/IFA | Yes | 47 | 24 | 39 | 54 | 2 | 5 | 28.50 | 21.81 | 17.45 |
| | No | 27 | 33 | 26 | 31 | 4 | 4 | 20.83 | 13.29 | 10.63 |
| Micro | Yes | 56 | 42 | 159 | 135 | 79 | 79 | 91.67 | 45.76 | 36.62 |
| emulsion | No | 11 | 4 | 17 | 16 | 3 | 2 | 8.83 | 6.74 | 5.39 |

VLP—artificial virus-like particles
CFA/IFA—complete Freund's adjuvant/incomplete Freund's adjuvant

An RCPL having 30 peptides, each 28 residues in length, that models the antigenic diversity of HIV-1 subtype B variants was synthesized in the form of MAP4. The antigenic properties of the RCPL was compared to a VCPL which also modeled the antigenic diversity of HIV-1 subtype B variants.

RCPL sequences (a period ‘.’ indicates that the residue unchanged from the first sequence listed):

NNNTRKSIHIGPGQAFYATGDIIGDIRQ
.....T..RL...RT..T..E......K
S.....GV.......W....Q.......
S.......R....R..FR......N...
.......V......V..T..E.T....K
.....RR.RM...RT.............
G.......T....R.L.T..E...N...
.....RG.P......L...........K
.....T.V.L...RT..T..Q.......
........RM..........G.T.N...
S.......P.....T....D.......K
......G......R.WHT..Q.......
S......V.L....V.........N...
.....TG......R..FT..E......K
G....R..R..L.R......E.......
.......TR......L.T....T.....
S......VT....RT..R.........K
.....T...L....V.F...E.......
......G.R..........D....N..K
......R..M...RT.....Q...N...
S......VT....R.L.T..G.......
.....R...L....VW.T..E.......
G.......R......L......T....K
.....RGV.....RT..T......N...
S....T.VP....R......Q......K
........RL.W..T..T.........K
..K..R..........H...........
S.....R..L...R...T..E...N...
..............T...R.Q.T.....
.....TG.P....R.W.T..Q.......

A rabbit was immunized 3 times with the MAP4 VCPL and CFA (first immunization) or IFA (second immunization). The antibody titer of the sera was tested by ELISA using either the immunizing antigen, or the MAP4 RCPL as the immobilized peptide. The titer of rabbit serum in ELISA with MAP4 VCPL on solid phase was 1:204,800, and with MAP4 RCPL on solid phase was 1:102,400.

A number of embodiments have been described. Nevertheless, it will be understood that various modifications may be made. For example, chimeric peptide libraries can be designed to mimic the antigenic diversity of other HIV proteins, or of proteins from other variable pathogens. Accordingly, other embodiments are within the scope of the following claims.

1-28. (canceled)

29. A composition comprising a family of antigenic peptides having amino acid sequences having antigenic similarity to amino acid sequences of a variable region of a pathogen protein, wherein each antigenic peptide in the family has at least one amino acid position that varies relative to other antigenic peptides in the family.

30. The composition of claim 29, wherein one amino acid residue occurs more frequently than another in the position that varies.

31. The composition of claim 29, wherein the family includes greater than 150 mutually unique antigenic peptides.

32. The composition of claim 29, wherein the family includes greater than 1,000 mutually unique antigenic peptides.

33. The composition of claim 29, wherein the family includes fewer than 100,000 mutually unique antigenic peptides.

34. The composition of claim 29, wherein the family includes fewer than 50,000 mutually unique antigenic peptides.

35. The composition of claim 29, wherein the family includes between 1,000 and 50,000 mutually unique antigenic peptides.

36. The composition of claim 29, wherein the pathogen protein is HIV gp120.

37. The composition of claim 36, wherein the variable region is selected from the group consisting of the V1 region, the V2 region, the V3 region, the V4 region and the V5 region.

38. The composition of claim 37, wherein the family of antigenic peptides includes sequences having antigenic similarity to sequences from a subtype of HIV.

39. The composition of claim 38, wherein the subtype is selected from the group consisting of subtype A, subtype B, subtype C, subtype D, subtype F, subtype G, a recombinant subtype, a subtype of HIV group N, a subtype of HIV group O, and combinations thereof.

40. The composition of claim 29, wherein at least two members of the family of antigenic peptides are mixed together.

41. The composition of claim 29, wherein the family of antigenic peptides are separated according to sequence.

42. The composition of claim 29, wherein the family includes a multiple antigenic peptide.

43-101. (canceled)

102. A peptide library comprising a family of peptides including the fragment:
—N—N—T—R—K—X⁴—I—X⁵—X⁶—G—X⁷—G—X⁸—X⁹—X¹⁰—X¹¹—X¹²—T—X¹³—X¹⁴—I—X¹⁵—G—X¹⁶—I—R—
wherein each X⁴-X¹⁶ is a fragment zero, one, two or three amino acid residues in length.

103. The peptide library of claim 102, wherein the family has antigenic similarity to the V3 region of HIV gp120.

104. The peptide library of claim 103, wherein the family has antigenic similarity to the V3 region of HIV gp120 of HIV subtype B.

105. The peptide library of claim 102, wherein the family of peptides have the formula:
X¹—C—X²—R—P—X³—N—N—T—R—K—X⁴—I—X⁵—X⁶—G—X⁷—G—X⁸—X⁹—X¹⁰—X¹¹—X¹²—T—X¹³—X¹⁴—I—X¹⁵—G—X¹⁶—I—R—X¹⁷—A—X¹⁸—C—X¹⁹
wherein each X¹-X¹⁹ is a fragment zero, one, two or three amino acid residues in length.

106. The peptide library of claim 105, wherein for each peptide of the family, X¹ independently is N, T, or H.

107. The peptide library of claim 105, wherein for each peptide of the family, X² independently is T or I.

108. The peptide library of claim 105, wherein for each peptide of the family, X³ independently is N, S, or G.

109. The peptide library of claim 105, wherein for each peptide of the family, X⁴ independently is S, G, or R.

110. The peptide library of claim 105, wherein for each peptide of the family, X⁵ independently is H, P, N, T or Y.

111. The peptide library of claim 105, wherein for each peptide of the family, X⁶ independently is I or M.

112. The peptide library of claim 105, wherein for each peptide of the family, X⁷ independently is P, L, or W.

113. The peptide library of claim 105, wherein for each peptide of the family, X⁸ independently is R, Q, G or S.

114. The peptide library of claim 105, wherein for each peptide of the family, X⁹ independently is A, V or T.

115. The peptide library of claim 105, wherein for each peptide of the family, X¹⁰ independently is F, W, or V.

116. The peptide library of claim 105, wherein for each peptide of the family, X¹¹ independently is Y, F or H.

117. The peptide library of claim 105, wherein for each peptide of the family, X¹² independently is T or A.

118. The peptide library of claim 105, wherein for each peptide of the family, X¹³ independently is G, E or R.

119. The peptide library of claim 105, wherein for each peptide of the family, X¹⁴ independently is E, Q, R or G.

120. The peptide library of claim 105, wherein for each peptide of the family, X¹⁵ independently is I or T.

121. The peptide library of claim 105, wherein for each peptide of the family, X¹⁶ independently is D or N.

122. The peptide library of claim 105, wherein for each peptide of the family, X¹⁷ independently is Q or K.

123. The peptide library of claim 105, wherein for each peptide of the family, X¹⁸ independently is H or Y.

124. The peptide library of claim 105, wherein for each peptide of the family, X¹⁹ independently is N or T.

125. The peptide library of claim 105, wherein for each peptide of the family, X¹ independently is N, T or H; X² independently is T or I; X³ independently is N, S, or G; X⁴ independently is S, G, or R; X⁵ independently is H, P, N, T or Y; X⁶ independently is I or M; X⁷ independently is P, L, or W; X⁸ independently is R, Q, G or S; X⁹ independently is A, V or T; X¹⁰ independently is F, W, or V; X¹¹ independently is Y, F or H; X¹² independently is T or A; X¹³ independently is G, E or R; X¹⁴ independently is E, Q, R or G; X¹⁵ independently is I or T; X¹⁶ independently is D or N; X¹⁷ independently is Q or K; X¹⁸ independently is H or Y; and X¹⁹ independently is N or T.

126. The peptide library of claim 105, wherein at least two members of the family of peptides are mixed together.

127. The peptide library of claim 105, wherein the family of peptides are separated according to sequence.

128. The peptide library of claim 105, wherein the family includes greater than 150 mutually unique peptide sequences.

129. The peptide library of claim 105, wherein the family includes fewer than 100,000 mutually unique peptide sequences.

130. The peptide library of claim 105, wherein the family includes fewer than 500 mutually unique peptide sequences, the sequences being representative of the entire sequence diversity available.

131-279. (canceled)
